(19) United States
(12) Patent Application Publication
(10) Pub. No.: US 2007/0256352 A1
(43) Pub. Date: Nov. 8, 2007

(54) Substituted hexahydro-1,4-diazepin-5-ones and compositions stabilized therewith

(76) Inventors: Mervin G. Wood, Maysville, KY (US); Andrea Roberts, Middletown, CT (US); Robert E. Detlefsen, Putnam Valley, NY (US)

Correspondence Address: JoAnn Villamizar;Ciba Specialty Chemicals Corp., Patent Department, 540 White Plains Road, P.O. Box 2005, Tarrytown, NY, 10591-9005, US

(21) Appl. No.: 11/789,740

(22) Filed: Apr. 25, 2007

Related U.S. Application Data
(60) Provisional application No. 60/797,167, filed on May 3, 2006.

(51) Int. Cl. C11C 5/00; C07D 243/02

(52) U.S. Cl. 44/275; 540/492

(57) ABSTRACT

The instant invention pertains to white, dyed, dipped, unscented and/or scented candle wax which is effectively stabilized against discoloration and fading by the incorporation therein of a substituted hexahydro-1,4-diazepin-5-ones in combination with a UV absorber and/or an antioxidant. Additionally, some novel substituted hexahydro-1,4-diazepin-5-one compounds are described.

This application claims the benefit under 35 U.S.C. 119(e) of U.S. Provisional Application No. 60/797,167, filed May 3, 2006, the contents of which are hereby incorporated by reference.

The instant invention pertains to white, dyed, dipped, unscented and/or scented candle wax which is effectively stabilized against discoloration and fading by the incorporation therein of a substituted hexahydro-1,4-diazepin-5-one in combination with a UV absorber and/or an antioxidant. Additionally, some novel substituted hexahydro-1,4-diazepin-5-one compounds are described.

### BACKGROUND

Candles have been known for many centuries going back to the eighth century B.C. The nature of candles is described in Ullmann's Encyclopedia of Industrial Chemistry, Volume A5 at pages 29-30 where it is seen that candles are made from paraffin, beeswax and stearin as basic materials, and where a host of additives may also be present.

It is not surprising that with candles and wax becoming increasingly more important attention was paid as to how to stabilize the said materials. At the National Candle Association Meeting in Houston, 1994, R. van der Vennet presented a paper on “Antioxidants in Wax—Replacement of BHT” touting the use of Vitamin E (tocopherol) as an antioxidant to prevent the yellowing of wax when oxidized. WO 94/13736 describes the same invention.

EP 359,488 A3 and EP 133,964 B1 describe stabilized waxes used in cosmetics where the waxes are the same or similar to those used in candles.

EP 5,922 A1 describes lip cosmetics where the waxes are useful in lipsticks and are related to those useful in candles.

U.S. Pat. Nos. 6,544,305 and 6,905,525 describe candle wax stabilized with piperazinones.

U.S. Pat. No. 5,879,694 describes in detail transparent gel candles both in composition and structure. The use of BHT as an antioxidant is mentioned.

At the National Candle Association Technical Meeting on Apr. 16, 1998, F. A. Ballentine et al., presented a paper entitled “Inhibiting Color Fading of Dyed Candles with CYASORB® Light Absorbers” in which the general theories of thermal oxidation and photodegradation are discussed along with data on the effect of light absorbers on color stability of dyed candle waxes. The light absorbers compared are 4-octyloxy-2-hydroxybenzophenone UV-531; 4-methoxy-2-hydroxybenzophenone UV-9; 2-(2-hydroxy-5-methylphenyl)-2H-benzotriazole UV-5365; 2-(2-hydroxy-5-tert-octylphenyl-2H-benzotriazole UV-5411 and 2-(2-hydroxy-3,5-di-tert-amylphenyl)-2H-benzotriazole UV-2337).

U.S. Pat. No. 5,964,905 teaches dyed and scented candle gels containing triblock copolymers and a hydrocarbon oil of high flash point. This reference teaches that a light (UV) absorber may be used to improve the shelf stability of the candle color when exposed to visible or ultraviolet light. Two preferred absorbers are ethylhexyl p-methoxycinnamate (PARSOL® MCX, Roche) and 2-(2-hydroxy-5-tert-octylphenyl)-2H-benzotriazole (CYASORB® 5411, Cytec).

WO 00/22037 teaches the stabilization of solid, shaped and colored wax articles, including candles, using a malonate UV absorber which may optionally contain a hindered amine moiety as part of the malonate compound structure. The wax articles are dyed with a variety of oil soluble dyes and pigments. The samples protected by dimethyl p-methoxybenzylidinemalonate exhibited better resistance to discoloration that did samples stabilized with selected benzotriazole or benzophenone UV absorbers.

Japanese Hei 3-278554 teaches that wax crayons (drawing materials) colored by organic pigments can be stabilized by a hindered amine and/or benzotriazole.

In respect to wax stabilization, the use of selected hindered amines and/or benzotriazole UV absorbers is also known in the prior art as seen in U.S. Pat. Nos. 3,530,084; 4,379,721; 4,616,051; 5,964,905; 6,221,115; and 6,296,674.

British 2,333,774 A and WO 00/31069 describe a number of 2-oxopiperazinyl derivatives useful for the stabilization of organic materials especially polymers and photographic materials.

U.S. Pat. No. 3,364,220 discloses the synthesis of 1,2,2,7,7-pentamethylhomopiperazin-4-one derivatives useful as an intermediate for hetereocyclic aminoalkylguanidines.

U.S. Pat. Nos. 3,962,255; and 3,966,711 disclose compositions containing 4-alkyl-1,4-diaza-5-oxo-cycloheptane and their 1-oxyls.

U.S. Pat. Nos. 4,167,512; 4,190,571; and 4,292,240 describe some 2-keto-1,4-diazacycloalkanes including piperazinones as light stabilizers.

U.S. Pat. No. 4,240,961 describes the preparation of piperazinones.

U.S. Pat. No. 6,117,995 depicts the preparation of sterically hindered amine ethers where the cyclic ring is inter alia a piperazinone. These compounds are useful as stabilizers.

Published United States Application No. US-2005-0261401-A1 discloses select hindered amine light stabilizers, the contents of which are herein incorporated by reference.

U.S. Pat. Nos. 4,033,928; 4,110,304 and 4,123,418 teach inter alia that selected piperazinones are useful in protecting epoxy resins and styrene polymers from degradation.

None of these references teach the superior performance provided when the substituted hexahydro-1,4-diazepin-5-one compounds are used in conjunction with a UV absorber and/or antioxidant to stabilize candle wax.

### SUMMARY

Disclosed is a stabilized candle wax composition comprising
(a) candle wax which is white and unscented; white and scented; dyed and unscented; dyed and scented; dipped and unscented; or dipped and scented, and
(b) an effective stabilizing amount of a combination of
   (i) a substituted hexahydro-1,4-diazepin-5-one compound which compound comprises one or more a moiety of formula (I) and

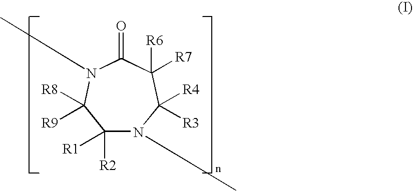

(ii) a UV absorber or an antioxidant, or a UV absorber and an antioxidant
wherein the ratio by weight of (i) to (ii) is from about 10:1 to about 1:10;

wherein
n is 1 or 2,

R₁, R₂, R₃ and R₄ are independently alkyl of 1 to 12 of carbon atoms, said alkyl substituted by one or two hydroxyl groups; or R₁ and R₂ together or R₃ and R₄ together or R₁ and R₂ together and R₃ and R₄ together are tetramethylene, pentamethylene or pentamethylene substituted by methyl;

R₆, R₇, R₈ and R₉ are independently hydrogen, alkyl of 1 to 12 of carbon atoms, said alkyl substituted by one or two hydroxyl groups; or R₆ and R₇ together or R₈ and R₉ together or R₆ and R₇ together and R₈ and R₉ together are tetramethylene, pentamethylene or pentamethylene substituted by methyl.

Also, disclosed and further subject of the invention are substituted hexahydro-1,4-diazepin-5-one compounds of formula (III) and (IV)

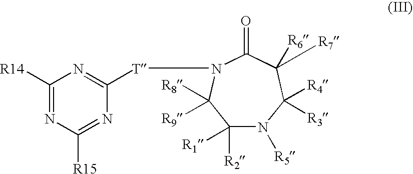

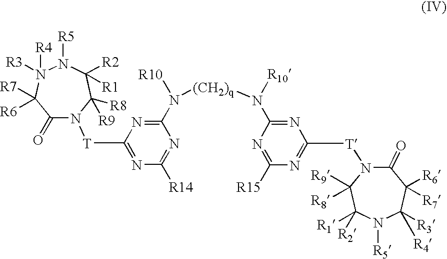

wherein
n is 1 or 2,
p is 1 to 12,
when n is 1,

G is hydrogen, alkyl of 1 to 18 carbon atoms, alkyl of 2 to 18 carbon atoms substituted by NH₂, by OH, by halogen, by —OR₁₀, by 4-hydroxy-3,5-di-tert-butylphenyl or by 4-hydroxy-3,5-dimethylphenyl, or by a mixture of these groups; or G is alkyl of 2 to 18 carbon atoms interrupted by —O—, by —CO—, by —NH— or by —NR₁₀— or by a mixture of said groups; or G is alkyl of 1 to 18 carbon atoms substituted by —COOR₁₁ or by phenyl;
when n is 2,

G is alkylene of 2 to 10 carbon atoms, alkenylene of 4 to 12 carbon atoms, alkylene of 3 to 15 carbon atoms substituted by one to four OH or said alkylene interrupted by one to four oxygen atoms; or G is phenylene, C₁-C₄alkylphenylene, —CO—(CH₂)ₚ—CO—, or —COO—;

R₁, R₂, R₃ and R₄ are independently alkyl of 1 to 12 of carbon atoms, said alkyl substituted by one or two hydroxyl groups; or R₁ and R₂ together or R₃ and R₄ together or R₁ and R₂ together and R₃ and R₄ together are tetramethylene, pentamethylene or pentamethylene substituted by methyl;

R₁′, R₂′, R₃′ and R₄′ and R₁″, R₂″, R₃″ and R₄″ are defined independently as for R₁, R₂, R₃ and R₄;

R₆, R₇, R₈ and R₉ are independently hydrogen, alkyl of 1 to 12 of carbon atoms, said alkyl substituted by one or two hydroxyl groups; or R₆ and R₇ together or R₈ and R₉ together or R₆ and R₇ together and R₈ and R₉ together are tetramethylene, pentamethylene or pentamethylene substituted by methyl;

R₆′, R₇′, R₈′ and R₈′ and R₆″, R₇″, R₈″ and R₉ ″ are defined independently as for R₆, R₇, R₈, and R₉;

R₅ is hydrogen, oxyl, hydroxyl, cyanoethyl, phenylalkyl of 7 to 15 carbon atoms, said phenylalkyl substituted on the phenyl ring by one to three radicals selected from alkyl of 1 to 4 carbon atoms and alkoxy of 1 to 4 carbon atoms, phenylalkoxy of 7 to 15 carbon atoms, said phenylalkoxy substituted on the phenyl ring by one to three radicals selected from alkyl of 1 to 4 carbon atoms and alkoxy of 1 to 4 carbon atoms, cycloalkoxy of 5 to 12 carbon atoms, alkenyl of 3 to 8 carbon atoms, alkynyl of 3 to 8 carbon atoms, alkoxy of 1 to 24 carbon atoms, alkyl of 1 to 24 carbon atoms, said alkyl substituted by one or two hydroxyl groups, said alkyl substituted by alkylcarbonyloxy of 2 to 18 carbon atoms, or said alkyl interrupted by one to ten oxygen atoms and substituted by one hydroxyl, by alkylcarbonyloxy of 2 to 18 carbon atoms or by 4-hydroxy-3,5-di-tert-butylbenzoyloxy; or R₅ is alkanoyl of 1 to 8 carbon atoms, alkenoyl of 3 to 5 carbon atoms, alkanoyloxy of 1 to 18 carbon atoms, glycidyl, or a group —CH₂CH(OH)-E where E is hydrogen, methyl or phenyl;

T, T′, and T″ are independently defined as alkylene of 1 to 12 carbon atoms; said alkylene interrupted by one to four oxygen atoms; said alkylene interrupted by one to four —NH— groups or —N(R₁₀)— groups; said alkylene interrupted by —OC(O)—, —C(O)—, —C(O)N(H)—, or —C(O)N(R₁₀)—; said alkylene substituted by one to four hydroxyl groups; said alkylene substituted by one to four —NH₂ groups or —N(R₁₀)H groups or —N(R₁₀)(R₁₃) groups; or T, T′, and T″ are independently —CH₂—CH(R₁₂)—O— wherein R₁₂ is hydrogen, methyl or phenyl, or T, T′, and T″ are independently —(CH₂)₃—NH— or T, T′, and T″ are independently a direct bond;

R₅′ and R₅″ are defined as for R₅;

R₁₀ and R₁₀′ are independently alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;

R₁₃ is alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;

R₆′ and R₆″ are defined as for R₆;

R₁₁ and R₁₁′ are independently hydrogen, alkyl of 1 to 18 carbon atoms, alkyl of 2 to 18 carbon atoms which is substituted by —NH₂, by —NHR₁₀, by —N(R₁₀)₂, by nitro, by hydroxy, by alkoxy of 1 to 18 carbon atoms or by a mixture of said groups; or R₁₁ and R₁₁′ are alkenyl of 3 to 18 carbon atoms, cycloalkyl of 5 to 12 carbon atoms, or said cycloalkyl which is substituted by alkyl of 1 to 4 carbon atoms or is interrupted by —O—, or R₁₁ and R₁₁′ is phenyl or phenylalkyl of 7 to 15 carbon atoms;

R₁₄ is —N(R₁₆)(R₁₆′) or is chlorine, alkoxy of 1 to 12 carbon atoms, thioalkyl of one to eighteen carbon atoms, 2-hydroxyethylamino or —N(R₁₁)(R₁₁′);

or R₁₄ is

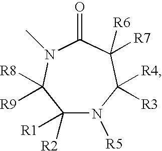

R₁₅ is defined as for R₁₄;

R₁₆ and R₁₆′ are independently glycidyl, C₂-C₁₂alkanoyl substituted by a di(C₁-C₆alkyl) phosphonate, or

R₁₆ and R₁₆′ are independently C₁-C₁₂alkyl, C₂-C₁₂alkanoyl or C₇-C₁₈phenylalkyl, each interrupted by one to six oxygen, sulfur or —N(R₁₀)— groups; C₁-C₁₂alkyl, C₂-C₁₂alkanoyl, phenyl or C₇-C₁₈phenylalkyl, each substituted by one to six hydroxy groups or by one to six —NHR₁₀ groups; C₂-C₁₂alkyl, C₂-C₁₂alkanoyl or C₇-C₁₈phenylalkyl, each interrupted by one to three —NR₁₀C(O)— groups; or R₁₆ and R₁₆′ are independently C₁-C₁₂alkyl, C₂-C₁₂alkanoyl, phenyl or C₇-C₁₈phenylalkyl, each substituted by one to three —SO₃H groups or by one to three —COOR₁₁ groups; and

q is2to8.

### DETAILED DISCLOSURE

For example, another embodiment of the instant invention, a substituted hexahydro-1,4-diazepin-5-one compound of component (b)(i) is a compound of formula (II), (III), or (IV)

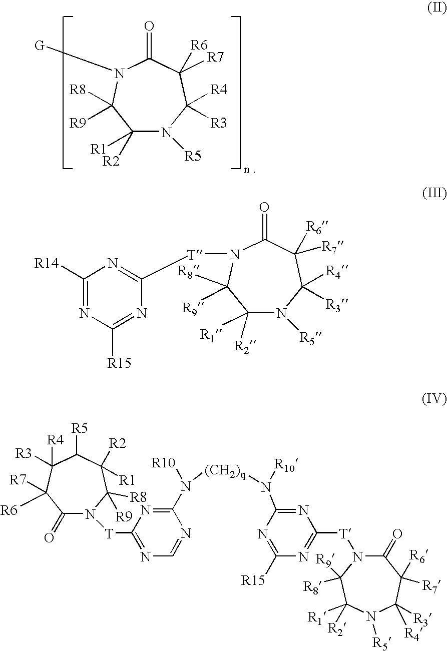

wherein
n is 1 or 2,
p is 1 to 12,
when n is 1,

G is hydrogen, alkyl of 1 to 18 carbon atoms, alkyl of 2 to 18 carbon atoms substituted by NH₂, by OH, by halogen, by —OR₁₀, by 4-hydroxy-3,5-di-tert-butylphenyl or by 4-hydroxy-3,5-dimethyl-phenyl, or by a mixture of these groups; or G is alkyl of 2 to 18 carbon atoms interrupted by —O—, by —CO—, by —NH— or by —NR₁₀— or by a mixture of said groups; or G is alkyl of 1 to 18 carbon atoms substituted by —COOR₁₁ or by phenyl;
when n is 2,

G is alkylene of 2 to 10 carbon atoms, alkenylene of 4 to 12 carbon atoms, alkylene of 3 to 15 carbon atoms substituted by one to four OH or said alkylene interrupted by one to four oxygen atoms; or G is phenylene, C₁-C₄alkylphenylene, —CO—(CH₂)ₚ—CO—, or —COO—;

R₁, R₂, R₃ and R₄ are independently alkyl of 1 to 12 of carbon atoms, said alkyl substituted by one or two hydroxyl groups; or R₁ and R₂ together or R₃ and R₄ together or R₁ and R₂ together and R₃ and R₄ together are tetramethylene, pentamethylene or pentamethylene substituted by methyl;

R₁′, R₂′, R₃′ and R₄′ and R₁″, R₂″, R₃″ and R₄″ are defined independently as for R₁, R₂, R₃ and R₄;

R₆, R₇, R₈ and R₉ are independently hydrogen, alkyl of 1 to 12 of carbon atoms, said alkyl substituted by one or two hydroxyl groups; or R₆ and R₇ together or R₈ and R₉ together or R₆ and R₇ together and R₈ and R₉ together are tetramethylene, pentamethylene or pentamethylene substituted by methyl;

R₆′, R₇′, R₈′ and R₈′ and R₆″, R₇″, R₈″ and R₉″ are defined independently as for R₆, R₇, R₈, and R₉;

R₅ is hydrogen, oxyl, hydroxyl, cyanoethyl, phenylalkyl of 7 to 15 carbon atoms, said phenylalkyl substituted on the phenyl ring by one to three radicals selected from alkyl of 1 to 4 carbon atoms and alkoxy of 1 to 4 carbon atoms, phenylalkoxy of 7 to 15 carbon atoms, said phenylalkoxy substituted on the phenyl ring by one to three radicals selected from alkyl of 1 to 4 carbon atoms and alkoxy of 1 to 4 carbon atoms, cycloalkoxy of 5 to 12 carbon atoms, alkenyl of 3 to 8 carbon atoms, alkynyl of 3 to 8 carbon atoms, alkoxy of 1 to 24 carbon atoms, alkyl of 1 to 24 carbon atoms, said alkyl substituted by one or two hydroxyl groups, said alkyl substituted by alkylcarbonyloxy of 2 to 18 carbon atoms, or said alkyl interrupted by one to ten oxygen atoms and substituted by one hydroxyl, by alkylcarbonyloxy of 2 to 18 carbon atoms or by 4-hydroxy-3,5-di-tert-butylbenzoyloxy; or R₅ is alkanoyl of 1 to 8 carbon atoms, alkenoyl of 3 to 5 carbon atoms, alkanoyloxy of 1 to 18 carbon atoms, glycidyl, or a group —CH₂CH(OH)-E where E is hydrogen, methyl or phenyl;

T, T′, and T″ are independently defined as alkylene of 1 to 12 carbon atoms; said alkylene interrupted by one to four oxygen atoms; said alkylene interrupted by one to four —NH— groups or —N(R₁₀)— groups; said alkylene interrupted by —OC(O)—, —C(O)—, —C(O)N(H)—, or —C(O)N(R₁₀)—; said alkylene substituted by one to four hydroxyl groups; said alkylene substituted by one to four —NH₂ groups or —N(R₁₀)H groups or —N(R₁₀)(R₁₃) groups; or T, T′, and T″ are independently —CH₂—CH(R₁₂)—O— wherein R₁₂ is hydrogen, methyl or phenyl, or T, T′, and T″ are independently —(CH₂)₃—NH— or T, T′, and T″ are independently a direct bond;

R₅′ and R₅″ are defined as for R₅;

R₁₀ and R₁₀′ are independently alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;

R₁₃ is alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;

R₆′ and R₆″ are defined as for R₆;

R₁₁ and R₁₁′ are independently hydrogen, alkyl of 1 to 18 carbon atoms, alkyl of 2 to 18 carbon atoms which is substituted by —NH₂, by —NHR₁₀, by —N(R₁₀)₂, by nitro, by hydroxy, by alkoxy of 1 to 18 carbon atoms or by a mixture of said groups; or R₁₁ and R₁₁′ are alkenyl of 3 to 18 carbon atoms, cycloalkyl of 5 to 12 carbon atoms, or said cycloalkyl which is substituted by alkyl of 1 to 4 carbon atoms or is interrupted by —O—, or R₁₁ and R₁₁′ is phenyl or phenylalkyl of 7 to 15 carbon atoms;

R₁₄ is —N(R₁₆)(R₁₆′) or is chlorine, alkoxy of 1 to 12 carbon atoms, thioalkyl of one to eighteen carbon atoms, 2-hydroxyethylamino or —N(R₁₁)(R₁₁′);

or R₁₄ is

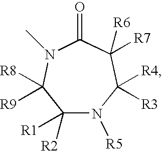

R₁₅ is defined as for R₁₄;

R₁₆ and R₁₆′ are independently glycidyl, C₂-C₁₂alkanoyl substituted by a di(C₁-C₆alkyl) phosphonate, or

R₁₆ and R₁₆′ are independently C₁-C₁₂alkyl, C₂-C₁₂alkanoyl or C₇-C₁₈phenylalkyl, each interrupted by one to six oxygen, sulfur or —N(R₁₀)— groups; C₁-C₁₂alkyl, C₂-C₁₂alkanoyl, phenyl or C₇-C₁₈phenylalkyl, each substituted by one to six hydroxy groups or by one to six —NHR₁₀ groups; C₂-C₁₂alkyl, C₂-C₁₂alkanoyl or C₇-C₁₈phenylalkyl, each interrupted by one to three —NR₁₀C(O)— groups; or R₁₆ and R₁₆′ are independently C₁-C₁₂alkyl, C₂-C₁₂alkanoyl, phenyl or C₇-C₁₈phenylalkyl, each substituted by one to three —SO₃H groups or by one to three —COOR₁₁ groups; and

q is 2 to 8.

Alkyl having up to 12 carbon atoms is branched or unbranched, and is for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl and dodecyl, 1,1,3,3,5,5-hexamethylhexyl.

Alkenyl is an unsaturated version of alkyl, and is branched or unbranched, for example isopropenyl, propenyl, hexenyl, heptenyl, and the like.

Unsubstituted or C₁-C₄alkyl-substituted C₅-C₈cycloalkyl is, for example, cyclopentyl, methyl-cyclopentyl, dimethylcyclopentyl, cyclohexyl, methylcyclohexyl, dimethylcyclohexyl, trimethyl-cyclohexyl, tert-butylcyclohexyl, cycloheptyl or cyclooctyl. For example cyclohexyl and tert-butylcyclohexyl.

C₁-C₄Alkyl-substituted phenyl, which contains for example 1 to 3, for instance 1 or 2, alkyl groups, is, for example, o-, m- or p-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2-methyl-6-ethylphenyl, 4-tert-butylphenyl, 2-ethylphenyl or 2,6-diethylphenyl.

Phenylalkyl includes substituted phenylalkyl, for example phenylalkyl substituted on the phenyl ring by from 1 to 3 C₁-C₄alkyl groups or from 1 to 3 halogen or by a mixture thereof, and is for example, benzyl, 4-chlorobenzyl, α-methylbenzyl, α,α-dimethylbenzyl, 2-phenylethyl, 2-methyl-benzyl, 3-methylbenzyl, 4-methylbenzyl, 2,4-dimethylbenzyl, 2,6-dimethylbenzyl or 4-tert-butylbenzyl.

Phenyl includes unsubstituted phenyl and phenyl substituted by from 1 to 3 C₁-C₄ alkyl groups or from 1 to 3 halogen or by a mixture thereof.

Alkanoyl having up to 12 carbon atoms is branched or unbranched, and is for example formyl, acetyl, propionyl, butanoyl, pentanoyl, hexanoyl, heptanoyl, octanoyl, nonanoyl, decanoyl, undecanoyl or dodecanoyl.

C₆-C₉Cycloalkylcarbonyl is, for example, cyclopentylcarbonyl, cyclohexylcarbonyl, cycloheptylcarbonyl or cyclooctylcarbonyl.

Carbamoyl of up to 12 carbon atoms is branched or unbranched, and is for example the carbamoyl equivalent of the alkanoyl groups, for example butamoyl, pentamoyl, hexamoyl and the like.

Alkenoyl of up to 12 carbon atoms is branched or unbranched, and is an unsaturated version of alkanoyl.

Benzoyl substituted by one to three C₁-C₄alkyl, is for example o-, m- or p-methylbenzoyl, 2,3-dimethylbenzoyl, 2,4-dimethylbenzoyl, 2,5-dimethylbenzoyl, 2,6-dimethylbenzoyl, 3,4-dimethylbenzoyl, 3,5-dimethylbenzoyl, 2-methyl-6-ethylbenzoyl, 4-tert-butylbenzoyl, 2-ethylbenzoyl, 2,4,6-trimethylbenzoyl, 2,6-dimethyl-4-tert-butylbenzoyl or 3,5-di-tert-butylbenzoyl.

C₂-C₂₅Alkanoyl substituted by a di(C₁-C₆alkyl) phosphonate group is, for example, (CH₃CH₂O)₂POCH₂CO—, (CH₃O)₂POCH₂CO—, (CH₃CH₂CH₂CH₂O)₂POCH₂CO—, (CH₃CH₂O)₂POCH₂CH₂CO—, (CH₃O)₂POCH₂CH₂CO—, (CH₃CH₂CH₂CH₂O)₂POCH₂CH₂CO—, (CH₃CH₂O)₂PO(CH₂)₄CO—, (CH₃CH₂O)₂PO(CH₂)₈CO— or (CH₃CH₂O)₂PO(CH₂)₁₇CO—.

C₂-C₁₂Alkyl interrupted by oxygen, sulfur or by —N(R₆)— is, for example, CH₃—O—CH₂—, CH₃—S—CH₂—, CH₃—N(CH₃)—CH₂—, CH₃—O—CH₂CH₂—O—CH₂—, CH₃—(O—CH₂CH₂—)₂O—CH₂—, CH₃—(O—CH₂CH₂—)₃O—CH₂— or CH₃—(O—CH₂CH₂—)₄O—CH₂—.

C₃-C₁₂Alkanoyl interrupted by oxygen, sulfur or by —N(R₆)— is, for example, CH₃—O—CH₂CO—, CH₃—S—CH₂CO—, CH₃—N(CH₃)—CH₂CO—, CH₃—O—CH₂CH₂—O—CH₂CO—, CH₃—(O—CH₂CH₂—)₂O—CH₂CO—, CH₃—(O—CH₂CH₂—)₃O—CH₂CO— or CH₃—(O—CH₂CH₂—)₄O—CH₂CO—.

Substitution by one to six also means of course by 1, 2, 3, 4, 5 or 6 groups, if the length of the chain so allows. Substitution by one to three means of course by 1, 2 or 3 groups, if the length of the chain allows.

Interruption by one to six groups means of course by 1, 2, 3, 4, 5 or 6 groups, if length of the chain allows.

C₇-C₁₈Phenylalkyl interrupted by oxygen, sulfur or by —N(R₆)— and unsubstituted or substituted on the phenyl radical by from one to three C₁-C₄alkyl groups is branched or unbranched, and is for example phenoxymethyl, 2-methyl-phenoxymethyl, 3-methyl-phenoxymethyl, 4-methyl-phenoxymethyl, 2,4-dimethyl-phenoxymethyl, 2,3-dimethyl-phenoxymethyl, phenylthiomethyl, N-methyl-N-phenyl-aminomethyl, N-ethyl-N-phenyl-aminomethyl, 4-tert-butyl-phenoxymethyl, 4-tert-butyl-phenoxyethoxy-methyl, 2,4-di-tert-butyl-phenoxymethyl, 2,4-di-tert-butyl-phenoxyethoxymethyl, phenoxyethoxyethoxyethoxymethyl, benzyloxymethyl, benzyloxyethoxymethyl, N-benzyl-N-ethyl-aminomethyl or N-benzyl-N-isopropyl-aminomethyl.

In another embodiment of the invention R₁, R₂, R₃ and R₄, R₁′, R₂′, R₃′ and R₄′ and R₁″, R₂″, R₃″ and R₄″ are defined independently and are each methyl, or R₁ and R₂ together and R₃ and R₄ together are pentamethylene.

In another embodiment of the invention, R₅, R₅′ and R₅″ are hydrogen or methyl.

Specific hexahydro-1,4-diazepin-5-one compounds of formula (II), (III), and (IV) of component (b) (i) which are useful in the instant invention include
(a) 1,3,5-tris(1-cyclohexyl-2,2,7,7-tetramethylhexahydro1,4-diazepin-5-on-4-yl)-s-triazine;
(b) 1,3,5-tris(1,2,2,7,7-pentamethylhexahydro1,4-diazepin-5-on-4-yl)-s-triazine;
(c) 1,2,2,7,7-pentamethylhexahydro-1,4-diazepin-5-one;
(d) 1,2-ethane-bis(1,2,2,7,7-pentamethylhexahydro1,4-diazepin-5-on-4-yl);
(e) 3,3,5,5-tetramethyl-7-oxo-[1,4]diazepine-1-carboxylic acid ethyl ester;
(f) 3,3,5,5-tetramethyl-7-oxo-[1,4]diazepine-1-carboxylic acid methyl amide;
(g) 3,3,5,5-tetramethyl-7-oxo-[1,4]diazepine-1-carboxylic acid hexyl amide;
(h) 4-benzyl-2,2,7,7-tetramethylhexahydro-1,4-diazepin-5-one;
(i) 1-hydroxyl-4-benzyl-2,2,7,7-tetramethylhexahydro-1,4-diazepin-5-one;
(j) 2,7-diethyl-1,2,3,7-tetramethylhexahydro-1,4-diazepin-5-one;
(k) 1-octyloxy-4-benzyl-2,2,7,7-tetramethylhexahydro-1,4-diazepin-5-one;
(l) 1-acetyl-2,2,7,7-tetramethylhexahydro-1,4-diazepin-5-one;
(m) 1-oxyl-4-benzyl-2,2,7,7-tetramethylhexahydro-1,4-diazepin-5-one;
(n) 1,3-bis(1-cyclohexyloxy-2,2,7,7-tetramethylhexahydro1,4-diazepin-5-on-4-yl)-5-(2-hydroxylethyl)amino-s-triazine;
(o) 1-(1-octyloxy-2,2,7,7-tetramethylhexahydro1,4-diazepin-5-on-4-yl)-3,5-bis(2-hydroxylethyl)amino-s-triazine;
(p) 1-(1-methoxy-2,2,7,7-tetramethylhexahydro1,4-diazepin-5-on-4-yl)-3,5-bis(methylthio)-s-triazine; or
(q) 1,3-bis(1-cyclohexyl-2,2,7,7-tetramethylhexahydro1,4-diazepin-5-on-4-yl)-5-(2-hydroxylethyl)amino-s-triazine.

Another embodiment of the instant invention is where the hexahydro-1,4-diazepin-5-one compounds of formula (II), (III), and (IV) of component (b)(i) are
(a) 1,3,5-tris(1-cyclohexyl-2,2,7,7-tetramethylhexahydro1,4-diazepin-5-on-4-yl)-s-triazine;
(b) 1,3,5-tris(1,2,2,7,7-pentamethylhexahydro1,4-diazepin-5-on-4-yl)-s-triazine;
(c) 1,3-bis(1-cyclohexyloxy-2,2,7,7-tetramethylhexahydro1,4-diazepin-5-on-4-yl)-5-(2-hydroxylethyl)amino-s-triazine;
(d) 1,3-bis(1-cyclohexyl-2,2,7,7-tetramethylhexahydro1,4-diazepin-5-on-4-yl)-5-(2-hydroxylethyl)amino-s-triazine;
(e) 1-(1-octyloxy-2,2,7,7-tetramethylhexahydro1,4-diazepin-5-on-4-yl)-3,5-bis(2-hydroxylethyl)amino-s-triazine; or
(f) 1-(1-methoxy-2,2,7,7-tetramethylhexahydro1,4-diazepin-5-on-4-yl)-3,5-bis(methylthio)-s-triazine.

In another embodiment of the invention, the ratio by weight of (i) to (ii) is from about 4:1 to about 1:4.

Another embodiment of the invention is where the ratio by weight of (i) to (ii) is from about 2:1 to about 1:2.

In still another embodiment of the invention, the effective amount of the UV absorber and antioxidant plus the hexahydro-1,4-diazepin-5-one compound [component (b)(i) plus component (b)(ii)] in the candle wax is from about 0.01 to about 10% by weight based on the wax.

In another embodiment of the invention the effective amount of the UV absorber plus hexahydro-1,4-diazepin-5-one compound in the candle wax is from about 0.1 to about 2% by weight based on the wax.

In still another embodiment of the invention the effective amount of the UV absorber plus hexahydro-1,4-diazepin-5-one compound in the candle wax is from about 0.1 to about 0.5% by weight based on the wax.

The UV absorber of component (ii) is a benzotriazole, a benzophenone, an α-cyanoacrylate, an oxanilide, an s-triazine, a cinnamate, a malonate, a benzoate or a salicylate, or a mixture thereof.

In another embodiment of the invention, the UV absorber is a benzotriazole, a benzophenone or an s-triazine.

Examples of the UV absorbers useful in the instant invention are
(a) 4-octyloxy-2-hydroxybenzophenone;
(b) 4-methoxy-2-hydroxybenzophenone;
(c) 2-(2-hydroxy-5-methylphenyl)-2H-benzotriazole;
(d) 2-(2-hydroxy-5-tert-octylphenyl-2H-benzotriazole;
(e) 2-(2-hydroxy-3,5-di-tert-amylphenyl)-2H-benzotriazole;
(f) octyl 3-(benzotriazol-2-yl)-5-tert-butyl-4-hydroxyhydrocinnamate;
(g) 2-(2-hydroxy-3,5-di-tert-butylphenyl)-2H-benzotriazole;
(h) 2-(2-hydroxy-5-tert-butylphenyl)-2H-benzotriazole;
(i) 2-(2-hydroxy-3,5-di-tert-butylphenyl)-2H-benzotriazole;
(j) 2-(2-hydroxy-3-tert-butyl-5-methylphenyl)-2H-benzotriazole;
(k) 2-(2-hydroxy-3-sec-butyl-5-tert-butylphenyl)-2H-benzotriazole;
(l) 2-(2-hydroxy-4-octyloxyphenyl)-2H-benzotriazole;
(m) 2-(2-hydroxy-3-dodecyl-5-methylphenyl)-2H-benzotriazole;
(n) 2-[2-hydroxy-3,5-di(α,α-dimethylbenzyl)phenyl]-2H-benzotriazole;
(o) 2-[2-hydroxy-3-(α,α-dimethylbenzyl)-5-tert-octylphenyl]-2H-benzotriazole;
(p) 2-{2-hydroxy-3-tert-butyl-5-[2-(omega-hydroxy-octa(ethyleneoxy)carbonyl)ethyl]-phenyl}-2H-benzotriazole;
(q) 2-{2-hydroxy-3-tert-butyl-5-[2-(octyloxy)carbonyl)ethyl]phenyl}-2H-benzotriazole;
(r) 2-ethylhexyl p-methoxycinnamate;
(s) 4-methoxy-2,2′-dihydroxybenzophenone;
(t) 4,4′dimethoxy-2,2′-dihydroxybenzophenone;
(u) 2,4-bis(2,4-dimethylphenyl)-6-(2-hydroxy-4-octyloxyphenyl)-s-triazine;
(v) 2,4-diphenyl-6-(2-hydroxy-4-hexyloxyphenyl)-s-triazine;
(w) 2,4-bis(2,4-dimethylphenyl)-6-[2-hydroxy-4-(3-do-/tri-decyloxy-2-hydroxypropoxy)-phenyl]-s-triazine;
(x) 2,4-bis(2,4-dimethylphenyl)-6-[2-hydroxy-4-(3-do-/tri-decyloxy-2-hydroxypropoxy)-5-α-cumylphenyl]-s-triazine;
(y) reaction product of 2,4,6-tris(2,4-dihydroxyphenyl)-s-triazine with octyl α-haloacetate; or
(z) the mixture of 3,3;3,5;5,5-methylene-bis[2,4-bis(2,4-dimethylphenyl)]-6-[2-hydroxy-4-(3-butyloxy-2-hydroxypropoxyphenyl)]-s-triazine.

Still other examples of UV absorbers useful in the instant invention are
(a) 4-octyloxy-2-hydroxybenzophenone;
(b) 4-methoxy-2-hydroxybenzophenone;
(d) 2-(2-hydroxy-5-tert-octylphenyl-2H-benzotriazole;
(o) 2-[2-hydroxy-3-(α,α-dimethylbenzyl)-5-tert-octylphenyl]-2H-benzotriazole;
(p) 2-{2-hydroxy-3-tert-butyl-5-[2-(omega-hydroxy-octa(ethyleneoxy)carbonyl)ethyl]-phenyl}-2H-benzotriazole;
(q) 2-{2-hydroxy-3-tert-butyl-5-[2-(octyloxy)carbonyl)ethyl]phenyl}-2H-benzotriazole;
(y) reaction product of 2,4,6-tris(2,4-dihydroxyphenyl)-s-triazine with octyl α-haloacetate; or
(z) the mixture of 3,3;3,5;5,5-methylene-bis[2,4-bis(2,4-dimethylphenyl)]-6-[2-hydroxy-4-(3-butyloxy-2-hydroxypropoxyphenyl)]-s-triazine.

Another embodiment of the instant invention involves a composition wherein the antioxidant is a phenolic antioxidant, phosphite, nitrone, amine oxide or hydroxylamine, or mixture thereof.

Examples of the above embodiment are those wherein the effective amount of UV absorber in combination with the hexahydro-1,4-diazepin-5-one compound and an antioxidant is from about 0.01 to about 10% by weight based on the wax.

Another embodiment is where the effective amount of UV absorber in combination with the hexahydro-1,4-diazepin-5-one compound and an antioxidant is from about 0.1 to about 2% by weight based on the wax.

Still another embodiment is where the effective amount of UV absorber in combination with the hexahydro-1,4-diazepin-5-one compound and an antioxidant is from about 0.1 to about 0.5% by weight based on the wax.

Examples of the antioxidants useful in this invention are
n-octadecyl 3,5-di-tert-butyl-4-hydroxyhydrocinnamate,
neopentanetetrayl tetrakis(3,5-di-tert-butyl-4-hydroxyhydrocinnamate),
di-n-octadecyl 3,5-di-tert-butyl-4-hydroxybenzylphosphonate,
1,3,5-tris(3,5-di-tert-butyl-4-hydroxybenzyl)isocyanurate,
thiodiethylene bis(3,5-di-tert-butyl-4-hydroxyhydrocinnamate),
1,3,5-trimethyl-2,4,6-tris(3,5-di-tert-butyl-4-hydroxybenzyl)benzene,
3,6-dioxaoctamethylene bis(3-methyl-5-tert-butyl-4-hydroxyhydrocinnamate),
2,6-di-tert-butyl-p-cresol,
2,2′-ethylidene-bis(4,6-di-tert-butylphenol),
1,3,5-tris(2,6-dimethyl-4-tert-butyl-3-hydroxybenzyl)isocyanurate,
1,1,3,-tris(2-methyl-4-hydroxy-5-tert-butylphenyl)butane,
1,3,5-tris[2-(3,5-di-tert-butyl-4-hydroxyhydrocinnamoyloxy)ethyl]isocyanurate,
3,5-di-(3,5-di-tert-butyl-4-hydroxybenzyl)mesitol,
hexamethylene bis(3,5-di-tert-butyl-4-hydroxyhydrocinnamate),
1-(3,5-di-tert-butyl-4-hydroxyanilino)-3,5-di(octylthio)-s-triazine,
N,N′-hexamethylene-bis(3,5-di-tert-butyl-4-hydroxyhydrocinnamamide),
calcium bis(ethyl 3,5-di-tert-butyl-4-hydroxybenzylphosphonate),
ethylene bis[3,3-di(3-tert-butyl-4-hydroxyphenyl)butyrate],
octyl 3,5-di-tert-butyl-4-hydroxybenzylmercaptoacetate,
bis(3,5-di-tert-butyl-4-hydroxyhydrocinnamoyl)hydrazide,
N,N′-bis[2-(3,5-di-tert-butyl-4-hydroxyhydrocinnamoyloxy)-ethyl]oxamide,
N,N-di-(C₁₂-C₂₄alkyl)-N-methyl-amine oxide, or
N,N-dialkylhydroxylamine prepared from di(hydrogenated tallow)amine by direct oxidation.

Still other embodiments of antioxidants useful in the instant invention are
neopentanetetrayl tetrakis(3,5-di-tert-butyl-4-hydroxyhydrocinnamate),
n-octadecyl 3,5-di-tert-butyl-4-hydroxyhydrocinnamate,
1,3,5-trimethyl-2,4,6-tris(3,5-di-tert-butyl-4-hydroxybenzyl)benzene,
1,3,5-tris(3,5-di-tert-butyl-4-hydroxybenzyl)isocyanurate,
2,6-di-tert-butyl-p-cresol, or
2,2′-ethylidene-bis(4,6-di-tert-butylphenol).

In another embodiment of the instant invention, the substituted hexahydro-1,4-diazepin-5-one compound of formula (III) and (IV) is selected from the group consisting of
(a) 1,3,5-tris(1-cyclohexyl-2,2,7,7-tetramethylhexahydro1,4-diazepin-5-on-4-yl)-s-triazine;
(b) 1,3,5-tris(1,2,2,7,7-pentamethylhexahydro1,4-diazepin-5-on-4-yl)-s-triazine;
(c) 1,3-bis(1-cyclohexyloxy-2,2,7,7-tetramethylhexahydro1,4-diazepin-5-on-4-yl)-5-(2-hydroxylethyl)amino-s-triazine;
(d) 1,3-bis(1-cyclohexyl-2,2,7,7-tetramethylhexahydro1,4-diazepin-5-on-4-yl)-5-(2-hydroxylethyl)amino-s-triazine;
(e) 1-(1-octyloxy-2,2,7,7-tetramethylhexahydro1,4-diazepin-5-on-4-yl)-3,5-bis(2-hydroxylethyl)amino-s-triazine; and
(f) 1-(1-methoxy-2,2,7,7-tetramethylhexahydro1,4-diazepin-5-on-4-yl)-3,5-bis(methylthio)-s-triazine.

Another embodiment of the present invention is stabilized compositions comprising
(c) an organic material subject to the deleterious effects of light, heat and oxygen, and
(d) an effective stabilizing amount of a present hexahydro-1,4-diazepin-5-one compound of formula (III) and (IV).

It should be noted that candles contain a host of various components. The base materials may be made up of the following:
paraffin wax,
natural oils,
polyamide plus fatty acid/ester,
fatty acids such as stearin,
opacifiers,
beeswax,
glycerides plus oxidized wax,
alcohols, and
ethylene oligomers.

Candles also contain a number of additives such as the following:
mold release agents,
fragrances,
insect repellants or insecticides,
hardeners,
crystal modifiers,
clarifiers,
guttering reducers,
colorants,
f.p. control agents,
stretchability improvers,
gelling agents,
extrusion aids, and
vortex reducers.

Each of the various components are meant to control or modify the properties of the candle to insure proper burning, reduce channeling, aid in uniform melting, and the like. The colorants and fragrances obviously are there to provide the proper color, scent or other aesthetic appeal.

Of increasing importance are the transparent gel candles which look like clear glass, but which burn like a classical candle. As is discussed in detail in U.S. Pat. No. 5,879,694, the relevant parts of which are incorporated herein by reference, these gel candles usually contain a copolymer selected from the group consisting of a tri-block, radial block, di-block or multi-block copolymer classically made up of at least two thermodynamically incompatible segments containing both hard and soft segments. Typical of such block copolymers is KRATON® (Shell Chemical Co.) which consists of block segments of styrene monomer units and rubber monomer or co-monomer units. The most common structure found in KRATON® D series is a linear ABA block with styrene-butadiene-styrene (SBS) or styrene-isoprene-styrene (SIS).

The following examples are meant for illustrative purposes only and are not to be construed to limit the instant invention in any manner whatsoever.

Raw Materials

Wax samples are supplied by the Candle-Lite Corporation. These samples contain dyes and fragrances.

The UV absorbers and hindered amine stabilizers are obtained from the Ciba Specialty Chemicals Corporation.

Sample Preparation

The wax samples obtained from the Candle-Lite Corporation already contain a dye and a fragrance (scent). In these cases, the wax is melted and the appropriate stabilizer(s) is (are) added and dissolved in the molten wax. The stabilized wax is then poured into five (5) 44 mm diameter aluminum pans giving five (5) wax disks.

Sample Exposure

Triplicate samples of each disk are exposed under a bank of six (6) cool-white fluorescent lamps (40 watts) or under a bank of six (6) UV lamps having a wavelength of 368 nm with the test samples being twelve (12) inches (30.48 cm) below the lamps.

Dye color fade (or color change) is measured by a Macbeth ColorEye Spectrophotometer with a 6 inch integrating sphere. The conditions are: 10 degree observer; D65 illuminant and 8 degree viewing angle.

Initial color measurements are taken using the above parameters. The L, a, and b values are calculated using the CIE system from the reflectance values. YI is calculated from the L, a and b values. Subsequent measurements are taken at specified intervals. Delta L, a, b and YI values are simply the difference between the initial values and the values at each interval. Delta(Δ) E is calculated as follows:

[(Delta L)²+(Delta a)²+(Delta b)²]^{1/2}=Delta E.

### EXAMPLE 1

### 1,2,2,7,7-Pentamethylhexahydro-1,4-diazepin-5-one

The title compound is an intermediate prepared following the general procedure of S. C. Dickerman and H. G. Lindwall, J. Org. Chem., 14, 530, (1949).

Other procedures useful for making this intermediate are given by M. W. Majchrzak et al., Tetrahedron, 37, 1075 (1981) and in U.S. Pat. No. 3,364,220 (see Example 10).

### EXAMPLE 2

### 1,3,5-Tris(1,2,2,7,7-pentamethylhexahydro1,4-diazepin-5-on-4-yl)-s-triazine

To a laboratory reactor are added 173 g (1.88 mol) of toluene, 18.9 g (0.26 mol) of N,N-dimethylformamide, and 10.3 g (0.056 mol) of 1,2,2,7,7-pentamethylhexahydro-1,4-diazepin-5-one, prepared in Example 1. The reaction mixture is refluxed for one hour. Sodium hydride (1.42 g, 0.059 mol) is added portionwise at 80 C. The reaction mass is then cooled to 0-5 C at which time 3.44 g (0.0186 mol) of cyanuric chloride is added portionwise over a ten minute period. The reaction mixture is stirred for 30 minutes at 20 C and is then heated to 90 C and held at that temperature for twelve hours. The reaction mixture is cooled to ambient and washed twice with water and once with saturated sodium chloride solution. Toluene is distilled off and the reaction mass is crystallized from ethanol. The title compound is obtained as (6.7 g, 57% yield) a white solid melting at 202-204 C.

Analysis:
Calcd. For C₃₃H₅₇N₉O₃: C, 63.12; H, 9.15; N, 20.08 Found: C, 63.15; H, 9.45; N, 19.90

### EXAMPLE 3

### 1,2-ethane-bis(1,2,2,7,7-pentamethylhexahydro1,4-diazepin-5-on-4-yl)

This compound is made by the general procedures given in U.S. Pat. No. 4,547,538.

### EXAMPLE 4

### Color Fade of Pink Scented Candle Wax Under UV Lamp Exposure

A variety of different stabilizers are evaluated in pink scented candle wax obtained from the Candle-Lite Corporation under uv lamp exposure. The ΔE values represent the change in color after the indicated days of exposure. A low ΔE value indicates less change in color and is highly desired.

| | ΔE after |
|---|---|
| Sample* (wt % add) | 37 days |
| Blank (no add) | 20.27 |
| D (0.3%) | 15.88 |
| B (0.15%) + C (0.15%) | 9.62 |
| A (0.15%) + D (0.15%) | 7.32 |
| A (0.15%) + M (0.15%) | 4.86 |
| F (0.15%) + C (0.15%) | 4.83 |
| G (0.15%) + H (0.15%) | 2.88 |
| A (0.15%) + E (0.15%) | 0.66 |

*A is octyl 3-(benzotriazol-2-yl)-5-tert-butyl-4-hydroxyhydrocinnamate, TINUVIN ® 384, CIBA.
B is 2-(2-hydroxy-5-tert-octylphenyl)-2H-benzotriazole, TINUVIN ® 329, CIBA.
C is 4-octyloxy-2-hydroxybenzophenone, CHIMMASORB ® 81, CIBA.
D is bis(1,2,2,6,6-pentamethylpiperidin-4-yl) sebacate, TINUVIN ® 292, CIBA.
E is 1,3,5-tris(1,2,2,7,7-pentamethylhexahydro-1,4-diazepin-5-on-4-yl)-s-triazine, Instant Example 2.
F is 1,2-ethane-bis(3,3,5,5-tetramethylpiperazin-2-on-1-yl), Goodrite UV 3034, Goodrich; a compound of U.S. Pat. No. 6,544,305.
G is 1,3,5-tris{N-cyclohexyl-N-[2-(2,2,6,6-tetramethylpiperazin-3-on-4-yl)ethyl]amino} -s-triazine, Goodrite 3150, Goodrich; a compound of U.S. Pat. No. 6,544,305.
H is 2,4-bis(2,4-dimethylphenyl)-6-(2-hydroxy-4-octyloxyphenyl)-s-triazine, UV 1164, Cytec.
M is 5-hydroxymethyl-3,3,5-trimethyl-2-morpholinone, Example 1 of U.S. Pat. No. 6,544,304.

These data show that a hexahydro-1,4-diazepin-5-one compound (Compound E) in combination with a UV absorber protects the pink scented candle wax from unwanted discoloration far better than conventional stabilizer systems.

### EXAMPLE 5

### Color Fade of Pink Scented Candle Wax Under UV Lamp Exposure

A variety of different stabilizers are evaluated in pink scented candle wax obtained from the Candle-Lite Corporation under UV lamp exposure. The ΔE values represent the change in color after the indicated days of exposure. A low ΔE value indicates less change in color and is highly desired.

| | ΔE after |
|---|---|
| Sample* (wt % add) | 44 days |
| Blank (no add) | 21.36 |
| D (0.3%) | 15.77 |
| B (0.15%) + C | 10.74 |
| (0.15%) | |
| A (0.15%) + D | 7.35 |
| (0.15%) | |
| I (0.15%) + J | 3.78 |
| (0.15%) | |
| E (0.15%) + A | 1.48 |
| (0.15%) | |

*A is octyl 3-(benzotriazol-2-yl)5-tert-butyl-4-hydroxyhydrocinnamate, TINUVIN ® 384, CIBA.
B is 2-(2-hydroxy-5-tert-octylphenyl)-2H-benzotriazole, TINUVIN ® 329, CIBA.
C is 4-octyloxy-2-hydroxybenzophenone, CHIMMASORB ® 81, CIBA.
D is bis(1,2,2,6,6-pentamethylpiperidin-4-yl) sebacate, TINUVIN ® 292, CIBA.
E is 1,3,5-tris(1,2,2,7,7-pentamethylhexahydro-1,4-diazepin-5-on-4-yl)-s-triazine, Instant Example 2.
I is 1,3,5-tris{N-cyclohexyl-N-[2-(1,2,2,6,6-pentamethylpiperazin-3-on-4-yl)ethyl]amino}-s-triazine, Goodrite 3159, Goodrich; a compound of U.S. Pat. No. 6,544,305.
J is 5-trifluoromethyl-2-(2-hydroxy-5-tert-octylphenyl)-2H-benzotriazole

These data show that a hexahydro-1,4-diazepin-5-one compound (Compound E) in combination with a UV absorber protects the pink scented candle wax from unwanted discoloration far better than conventional stabilizer systems.

### EXAMPLE 6

### Color Fade of Pink Scented Candle Wax Under UV Lamp Exposure

A variety of different stabilizers are evaluated in pink scented candle wax obtained from the Candle-Lite Corporation under UV lamp exposure. The ΔE values represent the change in color after the indicated days of exposure. A low ΔE value indicates less change in color and is highly desired.

| | ΔE after |
|---|---|
| Sample* (wt % add) | 32 days |
| Blank (no add) | 21.10 |
| D (0.3%) | 16.24 |
| B (0.15%) + C | 11.15 |
| (0.15%) | |
| A (0.15%) + D | 9.84 |
| (0.15%) | |
| K (0.15%) + L | 7.30 |
| (0.15%) | |

*A is octyl 3-(benzotriazol-2-yl)-5-tert-butyl-4-hydroxyhydrocinnamate, TINUVIN ® 384, CIBA.
B is 2-(2-hydroxy-5-tert-octylphenyl)-2H-benzotriazole, TINUVIN ® 329, CIBA.
C is 4-octyloxy-2-hydroxybenzophenone, CHIMMASORB ® 81, CIBA.
D is bis(1,2,2,6,6-pentamethylpiperidin-4-yl) sebacate, TINUVIN ® 292, CIBA.
K is 4-(3,5-dimethyl-4-hydroxybenzyl)-2,2,6,6-tetramethyl-3,5-diketopiperazine, Example 5e of U.S. Pat. No. 3,969,316.
L is C20–C40alkyl 3-(5-chloro-2H-benzotriazol-2-yl)-5-tert-butyl-4-hydroxyhydrocinnamate.

These data show that a hexahydro-1,4-diazepin-5-one compound (Compound E) in combination with a UV absorber protects the pink scented candle wax from unwanted discoloration far better than conventional stabilizer systems.

### EXAMPLE 7

### Color Fade of Pink Scented Candle Wax Under UV Lamp Exposure

A variety of different stabilizers are evaluated in pink scented candle wax obtained from the Candle-Lite Corporation under UV lamp exposure. The ΔE values represent the change in color after the indicated days of exposure. A low ΔE value indicates less change in color and is highly desired.

| | ΔE after |
|---|---|
| Sample* (wt % add) | 32 days |
| Blank (no add) | 21.10 |
| D (0.3%) | 16.24 |
| B (0.15%) + C | 11.15 |
| (0.15%) | |
| A (0.15%) + D | 9.84 |
| (0.15%) | |
| N (0.15%) + O | 5.97 |
| (0.15%) | |
| N (0.15%) + C | 4.73 |
| (0.15%) | |

*A is octyl 3-(benzotriazol-2-yl)-5-tert-butyl-4-hydroxyhydrocinnamate, TINUVIN ® 384, CIBA.
B is 2-(2-hydroxy-5-tert-octylphenyl)-2H-benzotriazole, TINUVIN ® 329, CIBA.
C is 4-octyloxy-2-hydroxybenzophenone, CHIMMASORB ® 81, CIBA.
D is bis(1,2,2,6,6-pentamethylpiperidin-4-yl) sebacate, TINUVIN ® 292, CIBA.
N is 3,3-dimethyl-1-oxa-4-azaspiro[4.5]decane, Example 1 of U.S. Pat. No. 6,540,795.
O is 2,4-bis(2,4-dimethylphenyl)-6-[2-hydroxy-4-(3-octyl/nonyl/decyloxy-2-hydroxypropoxy)-5-alpha-cumylphenyl]-s-triazine.

These data show that a hexahydro-1,4-diazepin-5-one compound (Compound E) in combination with a UV absorber protects the pink scented candle wax from unwanted discoloration far better than conventional stabilizer systems.

### EXAMPLE 8

Blue scented candle wax is well stabilized by 0.1% by weight of 2,4-bis(2,4-dimethylphenyl)-6-[2-hydroxy-4-(3-octyl/nonyl/decyloxy-2-hydroxypropoxy)-5-alpha-cumylphenyl]-s-triazine, 0.1% by weight of 1,3,5-tris(1-cyclohexyl-2,2,7,7-tetramethylhexahydro-1,4-diazepin-5-on-4-yl)-s-triazine and 0.1% by weight of N,N-dialkylhydroxylamine prepared from di(hydrogenated tallow)amine by direct oxidation.

### EXAMPLE 9

Green scented candle wax is well stabilized by 0.2% by weight of dimethyl p-methoxy-benzylidenemalonate, SANDUVOR® PR 25, in combination with 0.2% by weight of 1,3,5-tris(1-cyclohexyl-2,2,7,7-tetramethylhexahydro1,4-diazepin-5-on-4-yl)-s-triazine.

### EXAMPLE 10

Yellow scented candle wax is well stabilized by 0.1% by weight of di(1,2,2,6,6-pentamethyl-piperidin-4-yl) p-methoxybenzylidenemalonate, SANDUVOR® PR 31, in combination with 0.1% by weight of 1,3,5-tris(1,2,2,7,7-pentamethylhexahydro1,4-diazepin-5-on-4-yl)-s-triazine.

### EXAMPLE 11

Red unscented candle wax is well stabilized by 0.1% by weight of dimethyl di(p-chlorophenyl)-methylenemalonate in combination with 0.1% by weight of 1,2-ethane-bis(1,2,2,7,7-pentamethylhexahydro1,4-diazepin-5-on-4-yl).

### EXAMPLE 12

Red unscented candle wax is well stabilized by 0.2% by weight of dimethyl di(p-methoxy-phenyl)methylenemalonate in combination with 0.2% by weight of 3,3,5,5-tetramethyl-7-oxo-[1,4]diazepine-1-carboxylic acid ethyl ester.

### EXAMPLE 13

Red unscented candle wax is well stabilized by 0.1% by weight of 4,4′-dimethoxy-2,2′-dihydroxybenzophenone in combination with 0.1% by weight of 3,3,5,5-tetramethyl-7-oxo-[1,4]diazepine-1-carboxylic acid methyl amide.

### EXAMPLE 14

Red unscented candle wax is well stabilized by 0.2% by weight of 4-tert-butylphenyl 2-hydroxy-benzoate in combination with 0.1% by weight of 3,3,5,5-tetramethyl-7-oxo-[1,4]diazepine-1-carboxylic acid hexyl amide.

### EXAMPLE 15

Yellow unscented candle wax is well stabilized by 0.2% by weight of 2-ethoxy-2-ethyloxanilide in combination with 0.1% by weight of 4-benzyl-2,2,7,7-tetramethylhexahydro-1,4-diazepin-5-one.

### EXAMPLE 16

Yellow unscented candle wax is well stabilized by 0.2% by weight of 3-hydroxyphenyl benzoate in combination with 0.2% by weight of 1-hydroxyl-4-benzyl-2,2,7,7-tetramethylhexahydro-1,4-diazepin-5-one.

### EXAMPLE 17

Blue unscented candle wax is well stabilized by 0.2% by weight of dodecyl 3-phenyl-3-methyl-α-cyanoacrylate in combination with 0.1% by weight of 2,7-diethyl-1,2,3,7-tetramethylhexahydro-1,4-diazepin-5-one.

### EXAMPLE 18

Blue unscented candle wax is well stabilized by 0.2% by weight of dodecyl 3,3-diphenyl-α-cyanoacrylate in combination with 0.2% by weight of 1-octyloxy-4-benzyl-2,2,7,7-tetramethylhexahydro-1,4-diazepin-5-one.

### EXAMPLE 19

Yellow scented candle wax is well stabilized by 0.2% by weight of 2-{2-hydroxy-3-tert-butyl-5-[2-(omega-hydroxy-octa(ethyleneoxy)carbonyl)ethyl]phenyl}-2H-benzotriazole, TINUVIN® 1130, CIBA, in combination with 2% by weight of 1-acetyl-2,2,7,7-tetramethylhexahydro-1,4-diazepin-5-one.

### EXAMPLE 20

Yellow scented candle wax is well stabilized by 0.1% by weight of 2-[2-hydroxy-3-(α,α-dimethyl-benzyl)-5-tert-octylphenyl]-2H-benzotriazole, TINUVIN® 928, CIBA, in combination with 0.2% by weight of 1-oxyl-4-benzyl-2,2,7,7-tetramethylhexahydro-1,4-diazepin-5-one.

### EXAMPLE 21

Red scented candle wax is well stabilized by 0.1% by weight of 2-(2-hydroxy-5-methylphenyl)-2H-benzotriazole, TINUVIN® P, CIBA, in combination with 0.1% by weight of 1,3-bis(1-cyclohexyloxy-2,2,7,7-tetramethylhexahydro1,4-diazepin-5-on-4-yl)-5-(2-hydroxylethyl)amino-s-triazine.

### EXAMPLE 22

Green scented candle wax is well stabilized by 0.1% by weight of octyl 3-(benzotriazol-2-yl)-5-tert-butyl-4-hydroxyhydrocinnamate, TINUVIN® 384, CIBA, in combination with 0.1% by weight of 1-(1-octyloxy-2,2,7,7-tetramethylhexahydro1,4-diazepin-5-on-4-yl)-3,5-bis(2-hydroxylethyl)amino-s-triazine.

### EXAMPLE 23

Green scented candle wax is well stabilized by 0.1% by weight of octyl 3-(benzotriazol-2-yl)-5-tert-butyl-4-hydroxyhydrocinnamate, TINUVIN® 384, CIBA, in combination with 0.1% by weight of 1-(1-methoxy-2,2,7,7-tetramethylhexahydro1,4-diazepin-5-on-4-yl)-3,5-bis(methylthio)-s-triazine.

### EXAMPLE 24

Blue scented candle wax is well stabilized by 0.1% by weight of 2-(2-hydroxy-5-tert-octylphenyl-2H-benzotriazole, TINUVIN® 329, CIBA, in combination with 0.1% by weight of 1,3-bis(1-cyclohexyl-2,2,7,7-tetramethylhexahydro1,4-diazepin-5-on-4-yl)-5-(2-hydroxylethyl)amino-s-triazine.

### EXAMPLE 25

Blue scented candle wax is well stabilized by 0.2% by weight of 4-octyloxy-2-hydroxybenzo-phenone in combination with 0.1% by weight of 1,3-bis(1-cyclohexyl-2,2,7,7-tetramethylhexahydro1,4-diazepin-5-on-4-yl)-5-(2-hydroxylethyl)amino-s-triazine.

### EXAMPLE 26

Red unscented candle wax is well stabilized by 0.2% by weight of 2-ethylhexyl p-methoxy-cinnamate in combination with 0.1% by weight of 1-(1-methoxy-2,2,7,7-tetramethylhexahydro1,4-diazepin-5-on-4-yl)-3,5-bis(methylthio)-s-triazine.

### EXAMPLE 27

Red unscented candle wax is well stabilized by 0.1% by weight of 4,4′-dimethoxy-2,2′dihydroxy-benzophenone in combination with 0.1% by weight of 4-octyloxy-3,3,5,5-tetramethyl-1-octadecylpiperazin-2-one.

### EXAMPLE 28

Pieces of 1.27 cm×20.32 cm×30.48 cm western red cedar panels having a fine radial cut are used to test a commercially available tung oil phenolic varnish (supplied by McCloskey). One half of each panel is coated with two coats of unstabilized varnish. An equal amount of varnish containing 5% by weight (based on resin solids) of test stabilizers is applied to the other half of the panel in two coats. After storage for two weeks at ambient temperature, the wood panels are exposed outdoors at an angle of 45° S for a period of eight months. The 60° gloss of each half of the panel is measured at the top, middle and bottom portion of the panel and averaged according to ASTM D 523. Due to the lack of homogeneity of wood substrates, the gloss retention of the same varnish tends to differ slightly from panel to panel. Thus, the application of an unstabilized control varnish to every panel allows for a more meaningful measurement of the improvement in gloss due to the presence of the instant test compound.

The panels stabilized by the instant compounds show excellent gloss retention after long exposure.

### EXAMPLE 29

### Aromatic Urethane Varnish

A sample of commercial aromatic urethane varnish (Flecto-Varathane #90) is tested by the same method described in Example 28. After outdoor exposure at an angle of 45° S for a period of five months, the 60° gloss retention values of unstabilized and stabilized portions of the panels are determined.

The panels stabilized by the instant compounds show excellent gloss retention.

### EXAMPLE 30

### White Two-Component Polyester Urethane Gloss Enamel

A white polyester is formulated as shown below:

| | Parts |
|---|---|
| Component I | |
| Desmophen 670-90 (polyester glycol, Mobay) | 132.4 |
| Titanium Dioxide | 198.6 |
| Cellosolve Acetate | 98.9 |
| Sand Mill | |
| Desmophen 670-90 | 94.98 |
| Flow Aid | 0.28 |
| Tertiary Amine | 0.015 |
| Cellosolve Acetate | 332.6 |
| Component II | |
| Desmodur N-100 (polyisocyanate, Mobay) | 93.9 |
| Cellosolve Acetate | 58.7 |

This material is spray applied at a dry film thickness of 1.5-2.0 mil onto Bonderite 40 cold rolled steel panels that have been previously primed with a commercial epoxy polyamide maintenance primer (Sherwin-Williams Tile Clad II). Prior to application, the instant test compounds are added to the paint. After ambient storage for two weeks, three panels of each formulation are exposed outdoors at an angle of 45° S for a period of nine months. Thereafter, 20° gloss retention is determined by ASTM D 523-80 at the top, middle and bottom portions of each panel. Thus, the average values for nine gloss retention measurements for each triplicate set of panels are obtained.

The panels stabilized by the instant compounds show excellent gloss retention.

### EXAMPLE 31

### Acrylic Alkyd Refinish Enamel

A commercially available acrylic alkyd enamel pigments with non-leafing aluminum pigment and tinted a light blue is stabilized with a benzotriazole UV absorber and an instant hindered amine test compound and is then spray applied onto Bonderite 40 panels primed with an alkyd primer. After the coating is allowed to cure at room temperature for 14 days, the panels are exposed outdoors at an angle of 45° S for a period of eight months. The 20° gloss of the exposed panels is measured.

The panels stabilized by the instant compounds show excellent gloss retention.

### EXAMPLE 32

### Medium Oil Alkyd Enamel

A medium oil alkyd enamel pigmented with a non-leafing aluminum pigment and tinted light blue is stabilized with a benzotriazole UV absorber and an instant hindered amine test compound and is then sprayed applied onto cold rolled steel panels primed with an epoxy primer. After the coating is allowed to cure at room temperature for two weeks, the panels are exposed for accelerated weathering in a Xenon Arc Weather-Ometer for 840 hours. The 20° gloss values of the panels are determined before and after exposure.

The panels stabilized by the instant compounds show excellent gloss retention.

### EXAMPLE 33

### Electrocoat Composition

A typical E-coat composition is prepared by adding the diglycidyl ether of bisphenol A, polyethylene oxide diol, bisphenol A and xylene to a flask and heating to 135° C. The catalyst dimethylbenzylamine in xylene is added and the temperature maintained at 143° C. for two hours. The weight per epoxy (WPE) is measured and a previously prepared crosslinker composed of 2,4-toluenediisocyanate, trimethylolpropane blocked with an alcohol is then added and temperature reduced to 100° C. The remaining epoxy groups are then capped with two different secondary amines, namely diketimine of diethylenetriamine and methylethanolamine, in phenyl cellosolve. The temperature is maintained for one hour at 110° C. and the crosslinker hexa-methylenediisocyanate blocked with an alcohol is added. The temperature is maintained near 100° C. for 30 minutes and the resin mixture is added to deionized water, surfactant and lactic acid to give a resin emulsion.

To this resin emulsion is added the instant hindered amine compound, additional epoxy resin, carbon black, dibutyltin oxide catalyst, titanium dioxide, lead silicate, water and UV absorber. After dispersion using a sand mill to achieve proper fineness, the mixture is incorporated into an electrocoat bath with water for electrocoating onto a metal substrate.

The steel coating electrocoated with the above E-coat resin composition to a thickness of 23-30 mm and cured for 20 minutes at a temperature of 176-201° C. A pigmented resin layer is coated thereover at a thickness of 20-51 mm using an acrylic coating composition in an organic solver, pigments and a UV absorber. The coated panels are then baked at 121-129° C. to cure the pigmented layer.

The panels are then exposed outdoors for four months. The panels containing the instant hindered amine compound, particularly when used with a UV absorber, provided excellent resistance to delamination of the E-coat layer from the metal substrate.

### EXAMPLE 34

### Abrasion Resistant Coating Compositions

A solution in isopropanol of 50% (by weight) of 1,6-hexanediol, 10% 3-methacryloyloxypropyl-trimethoxysilane and 40% colloidal silica (in form of a 34% aqueous dispersion) is vacuum stripped to remove volatiles and combined with an instant hindered amine compound, a benzotriazole UV absorber and 2,4,6-trimethylbenzoyldiphenylphosphine photoinitiator. These compositions show no gelation on storage.

The compositions above are applied by roller coating to a 15 mil film of bisphenol A polycarbonate and the coated films are passed under a mercury lamp at 43° C. at a line speed of 610 cm/min. The compositions are cured to a colorless and optically clear coatings over the polycarbonate substrate.

The coatings as measured by the Taber Abrasion Test (ASTM D1044) are abrasion resistant.

The test specimens are also subjected to accelerated aging tests using an Atlas Ci35A Xenon Arc Weather-Ometer. The results show that the coatings containing the instant hindered amine compound exhibit excellent resistance to yellowness and haze formation.

### EXAMPLE 35

### Coating Over Polycarbonate

A two-component polyester urethane coating is stabilized by the addition of an instant hindered amine compound. The high-solids polyester polyol (Desmophen 670-80, Bayer) is crosslinked with an isocyanate based resin (Desmodue N-3390, Bayer). The coating is catalyzed with 0.015% by weight of dibutyltin dilaurate catalyst.

Plaques of polycarbonate-based plastic substrate (Xenoy) 4″×6″ are coated with the formulated clear coat at a thickness of approximately 1.5 mils. The coating is spray applied to the substrate and then baked at 82° C. for 20 minutes.

After storage for one week at room temperature, each plaque is cut into 2″×3″ strips with five replicates being made for each formulation. Each strip is placed into a 8-oz jar along with 2 mL of distilled water and sealed. All samples are placed in an over at 54° C. A crosshatch adhesion test is performed once a week on at least two of the replicate samples until the sample failed (5% adhesion loss) or until 40 days elapses.

The samples containing the instant hindered amine compounds exhibit excellent resistance to delamination.

### EXAMPLE 36

### Photographic Layers

Chromogenic photographic layers are prepared by hand-coating a gelatine emulsion containing silver bromide, yellow coupler and an additive on a polyethylene-coated paper.

The composition of the layer is as given in following table, amounts are in mg/m²:

| Component | Amount in the layer |
|---|---|
| Gelatine | 5150 |
| AgBr | 520 |
| Yellow coupler | 1.07 mmol/m² |
| Coupler solvent solv1 | 33% of the coupler weight/m² |
| Additive | 30% of the coupler weight/m² |
| Hardener ha1 | 300 |
| Surfactant su1 | 340 |

The layers are dried for 7 days in a ventilated cabinet.

The dried samples are exposed to white light through a stepwedge of 0.3 log E exposure steps. They are developed with the P94 process for negative color paper from Agfa-Gevaert, following the manufacturers' recommendations.

After exposure and processing, the remission density of the yellow dye is measured in the blue channel. The samples are then exposed in an Atlas WeatherOmeter behind a separate UV filter so as to receive 60 kJ/cm² light energy. The UV filter consists of an emulsion coated onto a polyester transparent support, such that the layer contains 1 g/m² of a benzotriazole UV absorber. The temperature is 43° C. and the relative humidity 50%. The density loss starting from a blue-density of 1 is determined. Low ΔD number are desired.

The additives of the present invention improve the light stability of yellow photographic layers.

### EXAMPLE 37

### Photographic Layers

Chromogenic photographic layers are prepared by hand-coating a gelatine emulsion containing silver bromide, yellow coupler and an additive on a polyethylene-coated paper.

The composition of the layer is as given in following table, amounts are in mg/m²:

| Component | Amount in the layer |
|---|---|
| Gelatine | 5150 |
| AgBr | 520 |
| Yellow coupler | 1.07 mmol/m² |
| Coupler solvent solv1 | 33% of the coupler weight/m² |
| Additive | 30% of the coupler weight/m² |
| Hardener ha1 | 300 |
| Surfactant su1 | 340 |

The layers are dried for 7 days in a ventilated cabinet.

The dried samples are exposed to white light through a stepwedge of 0.3 log E exposure steps. They are developed with the P94 process for negative color paper from Agfa-Gevaert, following the manufacturers recommendations.

After exposure and processing, the remission density of the yellow dye is measured in the blue channel. The samples are then subjected to storage in a Weiss climatic cabinet for 28 days. The density loss starting from a blue-density of 1 is determined. Low DD numbers are desired. The additives of the present invention improve the dark stability of yellow photographic layers.

### EXAMPLE 38

Molded test specimens are prepared by injection molding thermoplastic olefin (TPO) pellets containing pigments, a phosphite, a phenolic antioxidant or hydroxylamine, a metal stearate, ultraviolet light absorbers or a hindered amine stabilizer (an Instant Compound) or a mixture of UV absorber and hindered amine stabilizer (an Instant Compound).

Pigmented TPO pellets are prepared from pure pigment or pigment concentrate, coadditives and commercially available TPO by mixing the components in a Superior/MPM 1″ single screw extruder with a general all-purpose screw (24:1 L/D) at 400° F. (200° C.), cooled in a water bath and pelletized. The resulting pellets are molded into 60 mil (0.006 inch), 2″×2″ plaques at about 375° F. (190° C.) on a BOY 30M Injection Molding Machine.

Pigmented TPO formulation composed of polypropylene blended with a rubber modifier where the rubber modifier is an in-situ reacted copolymer or blended product containing copolymers of propylene and ethylene with or without a ternary component such as ethylidene norbornene are stabilized with a base stabilization system consisting of an N,N-dialkylhydroxylamine or a hindered phenolic antioxidant with or without an organophosphorus compound.

All additive and pigment concentrations in the final formulation are expressed as weight percent based on the resin.

Formulation contained thermoplastic olefin pellets and one or more of the following components:
0.0 to 2.0% pigment,
0.0 to 50.0% talc,
0.0 to 0.1% phosphite,
0.0 to 1.25% phenolic antioxidant,
0.0 to 0.1% hydroxylamine
0.05 to 0.10 calcium stearate,
0.0 to 1.25% UV absorber
0.0 to 1.25% hindered amine stabilizer.

The components are dry-blended in a tumble dryer prior to extrusion and molding.

Test plaques are mounted in metal frames and exposed in an Atlas Ci65 Xenon Arc Weather-Ometer at 70° C. black panel temperature, 0.55 W/m² at 340 nanometers and 50% relative humidity with intermittent light/dark cycles and water spray (Society of Automotive Engineers—SAE J 1960 Test Procedure). Specimens are tested at approximately 625 kilojoule intervals by performing color measurements on an Applied Color Systems spectrophotometer by reflectance mode according to ASTM D 2244-79. Data collected include delta E, L*, a* and b* values. Gloss measurements are conducted on a BYK-Gardner Haze/Gloss Meter at 60° according to ASTM D 523.

UV Exposure Testing

Test specimens exposed to UV radiation exhibit exceptional resistance to photodegradation when stabilized with light stabilizer systems comprising a combination of 2-(2-hydroxy-3,5-di-tert-amylphenyl)-2H-benzotriazole, an instant stabilizer and N,N′,N″,N′″-tetrakis[4,6-bis(butyl-(1,2,2,6,6-pentamethylpiperidin-4-yl)amino)-s-triazin-2-yl]-1,10-diamino-4,7-diazadecane. The control sample consists of a stabilizer formulation commonly used in the industry to impart UV stability. All of the samples contain a pigment, Pigment Red 177, and talc.

The test plaques described earlier contain the following (all concentrations are weight percent based on resin):

Polymer substrate is commercially available polyolefin blend POLYTROPE® TPP 518-01 supplied by A. Schulman Inc. Akron, Ohio)

Color package is 0.025% Red 3B—Pigment Red 177, C.l. #65300.

Each plaque contains:
0.2% 2-(3,5-di-t-amyl-2-hydroxyphenyl)-2H-benzotriazole;
0.1% calcium stearate; and
15% talc.

The Control plaques additionally contain
0.1% 50:50 blend of neopentanetetrayl tetrakis(4-hydroxy-3,5-di-tert-butylhydro-cinnamate) and [tris-(2,4-di-tert-butylphenyl)phosphite;
0.2% (bis(2,2,6,6-tetramethylpiperidin-4-yl)sebacate];
0.2% [polycondensation product of 4,4′-hexamethylene-bis(amino-2,2,6,6-tetramethyl-piperidine) and 2,4-dichloro-6-tert-octylamino-s-triazine].

The test plaques containing the instant stabilizers additionally each contain 0.05% N,N,-dialkyl-hydroxylamine;

The instant stabilizers demonstrate greatly improved gloss retention compared to the less effective control system. Resistance to color change upon UV exposure is also enhanced.

Polymer blends containing an unsaturated ternary component, such as EPDM blends, are especially benefited with the more efficient instant light stabilizer systems described above.

### EXAMPLE 39

Molded test specimens are prepared by injection molding thermoplastic olefin (TPO) pellets containing the instant compounds, pigments and other coadditives as described in Example 38.

The light stable formulations are painted with one-pack paint systems and tested for TPO/paint interactions. Before painting, the test specimens are first washed in accordance with GM998-4801 and dried for 15 minutes at 200° F. (94° C.). Adhesion promoter is applied to the dry film thickness of 0.2-0.4 mils. The samples are dried for five minutes before a 1K basecoat is applied to a film thickness of 1.2-1.4 mils. The painted panels are dried for three minutes, a clearcoat is then applied to a dry film thickness of 1.2-1.5 mils followed by ten minutes flash drying and a 30 minute oven bake at 250° F. (121° C.).

Paint adhesion is measured by Aggressive Adhesion Testing (proprietary test procedure conducted at Technical Finishing, Inc.) and Taber Scuff. Painted panels which retain greater than 80% of the paint finish are considered acceptable. After Aggressive Adhesion Testing, samples with less than 5% paint loss are deemed acceptable.

The instant compounds provide very low levels of paint loss when analyzed by the testing protocols listed above.

### EXAMPLE 40

Molded test specimens are prepared by injection molding polypropylene pellets containing pigments, a phosphite, a phenolic antioxidant or hydroxylamine, a metal stearate, ultraviolet light absorbers or a hindered amine stabilizer (an Instant Compound) or a mixture of UV absorbers and hindered amine stabilizers (Instant Compound).

Pigmented polypropylene pellets are prepared from pure pigment or pigment concentrates, stabilizers, co-additives and commercially available polypropylene by mixing the components in a Superior/MPM 1″ single screw extruder with a general all-purpose screw (24:1 L/D) at 475° F. (250° C.), cooled in a water bath and pelletized. The resulting pellets are molded into 60 mil (0.06 inch thick) 2″×2″ plaques at about 475° F. (250° C.) on a BOY 30M Injection Molding Machine.

Pigmented polypropylene formulations composed of polypropylene homopolymer or polypropylene copolymer are stabilized with a base stabilization system consisting of an N,N-dialkylhydroxylamine or a hindered phenolic antioxidant with or without an organophosphorus compound.

All additive and pigment concentrations in the final formulations are expressed as weight percent based on the resin.

Formulations contained polypropylene pellets and one or more of the following components;

0.0%-2.0% pigment,

0.0%-50.0% talc,

0.0%-50.0% calcium carbonate,

0.0%-0.1% phosphate,

0.0%-1.25% phenolic antioxidant,

0.0%-0.1% hydroxylamine,

0.05%-0.10% calcium stearate,

0.0%-1.25% UV absorber,

0.0%-1.25% hindered amine stabilizer.

The components are dry blended in a tumble dryer prior to extrusion and molding.

Test plaques are mounted in metal frames and exposed in an Atlas Ci65 Xenon Arc Weather-o-meter at 70° C. black panel temperature, 0.55 W/m² at 340 nanometers and 50% relative humidity with intermittent light/dark cycles and water spray (Society of Automotive Engineers—SAE J 1960 Test Procedure). Specimens are tested at approximately 625 kilojoule intervals by performing color measurements on an Applied Color Systems spectrophotometer by reflectance mode according to ASTM D 2244-79. Data collected included delta E, L*, a* and b* values. Gloss measurements are conducted on a BYK-GARDNER Haze/Gloss Meter at 60° according to ASTM D523.

UV Exposure Testing

Test specimens exposed to UV radiation exhibit exceptional resistance to photodegradation when stabilized with light stabilizer systems comprised of a combination of 2-(3,5-di-t-amyl-2-hydroxyphenyl)-2H-benzotriazole, an instant stabilizer, and an oligomeric hindered amine. The oligomeric hindered amine is oligomer of N-{[2-(N-2,2,6,6-tetramethylpiperidin-4-yl)butylamino]-s-triazin-4-yl}-N,N′-bis(2,2,6,6-tetramethylpiperidin-4-yl)-1,6-hexanediamine terminated with 2,4-bis(dibutylamino)-s-triazin-6-yl. The Control sample consists of a stabilizer formulation commonly used in the industry to impart UV stability. All of the samples contain Pigment Red 177.
All formulations are base stabilized with 0.05% dialklyhydroxylamine in the final resin formulation.
Polymer substrate is a commercially available polypropylene homopolymer—Profax 6501 (commercial supplier Montell Polyolefins).
Color package is 0.25% Red 3B—Pigment Red 177, C.I. #65300 in the final resin formulation.
Each formulation contains a hydroxyphenyl benzotriazole UV absorber—2-(2-hydroxy-3,5-di-tert-amylphenyl)-2H-benzotriazole.
Each formulation contains 0.1% calcium stearate.
Samples are 60 mil thick 2″×2″ injection molded plaques.
UV exposures conducted under SAE J 1960—Exterior Automotive conditions.

All additive and pigment concentrations in the final formulations are expressed as weight percent on the resin.

The light stabilized formulations show much greater resistance to photodegradation than unstabilized specimens which fail quickly under the UV exposure conditions outlined above.

### EXAMPLE 41

Fiber samples are prepared by extruding fiber-grade polypropylene with the instant compounds, coadditives and pigments. Typical formulations contain the instant compounds at levels from 0.05 to 2.0%, a metal stearate such as calcium stearate at 0.05 to 0.5%, pigments from 0 to 5%, UV absorbers at levels of 0.05 to 2.0%, phosphites at 0 to 0.1%, phenolic antioxidants at 0 to 1.25%, N,N-dialkylhydroxylamines at 0 to 0.1% and optionally other hindered amines (Instant Compounds) at levels of 0 to 2.0%. All additive and pigment concentrations in the final formulations are given as weight percent based on the resin.

Pigment concentrates are prepared from pure pigment and polypropylene (PROFAX®, Hercules) by mixing the two components in a high shear mixer in a ratio of 25% pigment and 75% resin, pressing the resulting resin/pigment mixture on a Wabash Compression molder (Model #30-1515-4T3) into a thin sheet and dividing the sheet into fine chips for dispersion in polypropylene at reduced concentrations. Alternatively, pigment concentrates are obtained as pigment dispersions in a suitable carrier resin for subsequent blending in fiber at reduced concentrations.

Formulations containing polypropylene, 0.05-0.1% phosphite, 0-1.25% phenolic antioxidant, 0-0.1% dialkylhydroxylamine, 0.05-0.1% calcium stearate, 0-1.25% UV absorber, 0-1.25% hindered amine are dry blended in a tumble dryer, extruded on a Superior/MPM 1″ single screw extruder with a general all-purpose screw (24:1 L/D) at 475° F. (246° C.), cooled in a water bath and pelletized. The resulting pellets are spun into fiber at about 475° F. (246° C.) on a HILLS Research Fiber Extruder (Model # REM-3P-24) fitted with a 41 hole, delta configuration spinneret. The spun tow is stretched at a draw ratio of 3.2:1 producing a final denier of 615/41.

Fiber samples are knitted into socks on a Lawson-HemphillFiber Analysis Knitter, cut into appropriate lengths and exposed in an Atlas Ci65 Xenon Arc Weather-O-meter at 89° C. black panel temperature, 0.55 W/m² at 340 nanometers and 50% relative humidity (Society of Automotive Engineers—SAE J 1885 Test Procedure).

Fiber samples are tested by performing color measurements on an Applied Color Systems spectrophotometer by reflectance mode according to ASTM D 2244-79. Identical, but separate, fiber samples are examined for catastrophic failure and the time to failure is recorded.

The samples containing the instant compounds exhibit good stabilization performance against the deleterious effects of UV light.

### EXAMPLE 42

### Glycidyl Methacrylate-Based Powder Clearcoat

One of the major new coating technologies that can be used to meet increasingly stringent VOC solvent emission requirements is the use of powder coatings. Applications requiring the use of light stabilizers include clearcoats for finishing of automotive topcoats, finishing of garden implements, protection of automotive wheel covers. For optimum incorporation and shelf stability, stabilizers used in a powder coating should be moderate-melting (˜100° C.) solids, nonvolatile, and heat stable at typical powder coating baking temperatures (140-180° C.).

Prior to incorporation of the Hindered Amine Light Stabilizers under test, a premix of commercially available GMA-based powder coating resin, UV absorber, and flow aids is made by extruding together at 145° C. The Hindered Amine Light Stabilizers under test are then incorporated into portions of this premix, along with a commercially available 1,12 dodecanoic acid crosslinking resin. The final mix is extruded at 100° C., then the extrudate is milled on an ultracentrifugal mill and powder cyclone, and sieved. The powders are electrostatically sprayed onto a basecoat to a film thickness of 60 microns. The coatings are cured for 30 minutes at 160° C.

The panels are weathered in a Xenon WeatherOmeter, and in Florida at 5° South angle. Gloss and color are measured.

The panels stabilized by the instant compounds show excellent gloss retention.

### EXAMPLE 43

### Oil Modified Urethane Alkyd for Wood Application

The Hindered Amine Light Stabilizers under test are incorporated into a commercially available solvent-borne urethane alkyd McWhorter 43-4355. A 2-hydroxy benzotriazole UV absorber is also incorporated into all formulations. The Hindered Amine light stabilizers are added at equivalent piperidine levels. After mixing, the clear coatings are applied by brushing to white pine boards. Each board is divided into 8 sections separated by a groove ⅛″ deep, which is sealed with a film-forming clear varnish and the back and sides are coated with a white chlorinated pool paint. The stabilized coatings are applied in triplicate in three coats to sections of the boards in such a way as to ensure that a control formulation is present on each of the boards for comparison. The wood samples are allowed to dry for 1 week, then placed on exposure in Florida, Australia, and New York.

The panels stabilized by the instant compounds show good color retention, cracking resistance, and visual gloss retention.

### EXAMPLE 44

### Preformed Films for Lamination to Plastic Parts

The instant invention also pertains to protective and decorative films which are preformed, then applied to a substrate via a dry paint transfer process. These films consist of a single decorative layer which is applied to a carrier sheet, then laminated to a self-supporting, thermoformable backing sheet. The carrier sheet is then removed from the opposite side of the film, exposing the decorative layer. The composite film/backing sheet then is thermoformed to a three-dimensional shape. Additionally, these films may also consist of multiple layers, where, for example, a thermoplastic, thermoformable clearcoat is applied to the carrier sheet, then hardened to form an optically clear film. A color coat is then applied to the exposed face of the clearcoat, and hardened, resulting in a clear coat/color coat paint film supported by the carrier. This composite is then laminated to a thermoformable backing sheet, as above. The carrier sheet is removed, as above, and the composite clearcoat/colorcoat/backing is then thermoformed, as above.

The polymeric resins for the above application must be thermoplastic, and may be fluoropolymer/acrylic blends.

### EXAMPLE 45

1 to 5 g of the instant compounds, 1 g of tris(2,4-di-tert-butylphenyl)phosphite, 0.5 g of pentaerythritol tetrakis (3-(3,5-di-tert-butyl-4-hydroxyphenyl)propionate) and 1 g of calcium stearate are mixed in a turbomixer with 1000 g of polypropylene powder having a melt index of 2.1 (measured at 230° C. and 2.16 Kg). The final concentration of hindered amine is 0.1% to 0.5% in the PP.

The mixture is extruded at 200-220° C. to give polymer granules which are subsequently converted to stretched tapes of 50 microns thickness and 2.5 mm width, using a semi industrial type of apparatus (Leonard-Sumirago(VA)-Italy) and working under the following conditions:

Extruder temperature: 210-230° C.

Head temperature: 240-260° C.

Stretch ratio: 1:6

The tapes thus prepared are mounted on a white card and expose in Weather-Ometer 65 WR (ASTM D 2565-85) with a black panel temperature of 63° C.

The residual tensile strength is measured, by means of a constant velocity tensometer, on a sample taken after various light exposure times; from this, the exposure time (in hours) required to halve the initial tensile strength (T₅₀) is measured.

The tapes containing the present hindered amines exhibit excellent UV light stability.

Water carry-over is expressed as the rate at which a film or stretched tape can be extruded before water from the water-cooling bath is carried along with the film. The higher the water carry-over value, the faster and more economically a film or stretched tape can be processed. It is well known in the art that additives can have an effect on water carry-over. The films containing the present hindered amines exhibit high production rates.

### EXAMPLE 46

Film grade polyethylene is dry blended with approximately 10% by weight of a test additive of the present invention and then melt compounded at 200° C. into “Masterbatch” pellets. The fully formulated “Masterbatch” pellets are dry blended with polyethylene resin to get the desired final stabilizer concentrations. Typical formulations contain an additive of the present invention at levels from 0.05% to 2.0%, a metal stearate such as calcium stearate at 0.05% to 0.5%, a phosphite at 0% to 0.1%, a phenolic antioxidant at 0% to 1.25%, an N,N-dialkylhydroxylamine at 0% to 0.1% and optionally a hindered amine at 0% to 2.0%. The stabilized fully formulated resin is then blown at 200° C. into a 150 micron thick film on a DOLCI film line.

The resulting films are exposed for 4 hours to 20 liters of an aqueous solution containing 22.5 ppm chlorine. The chlorine is made available via Leslies Fast Dissolving Super Shock—Super Chlorinator (Shock and Algae control) from OLIN Pool Products, Norwalk Conn. This Super Shock is 78% Calcium Hypochlorite is used accordingly to make the 22.5 ppm Cl available. After the 4 hours of the chlorine exposure the samples are rinsed in distilled water 3 times, and air-dried to prepare them for accelerated weathering. A duplicate sample is exposed to distilled water without the Chlorine. All the dipped samples are exposed for 250 hour intervals in a Weather-O-meter 65 WR (ASTM D 2565-85-dry) with a black panel temperature of 63° C. After each 250 hour interval of accelerated weathering, the samples are again exposed to the aqueous exposure as above. Failure is defined as the time to a 50% loss of original elongation. This test is designed to simulate exposure to pool chemicals as would be experienced by pool covers.

The films containing the present compounds show good resistance to pool chemicals containing chlorine.

Other polyolefin articles, such as pool hoses, exposed to pool chemicals containing the present compounds show good resistance to pool chemicals containing chlorine.

### EXAMPLE 47

100 parts high density polyethylene are dry blended with 0.4 parts of 1,2-bis(3,5-di-tert-butyl-4-hydroxyhydrocinnamoyl)hydrazine and 0.2 parts of the instant hindered amines. The mixtures are melt compounded into pellets at 230° C. in a Superior/MPM extruder using a 24:1 L/D screw with Maddock mixing head at 60 rpm.

The pelletized polyethylene containing the stabilizer mixtures are compression molded at 400° F. into 10 mil (0.01 inch) thick films with Mylar backing. “Initial oxidation induction time” (OIT) is measured on these test films.

The sample films are then submersed in Witcogel®, available from Witco, a typical hydrocarbon cable filler grease used in telecom cables. The Witco filling compound contains 0.6% thiodiethylene bis[3-(3,5-di-tert-butyl-4-hydroxyphenyl)propionate]. The sample films submersed in the filling compound are exposed in an air oven at 70° C. for 14 days. The samples are then wiped clean of the cable filler grease. “Aged oxidation induction time” is measured on these samples.

OIT testing is accomplished using a differential scanning calorimeter as per ASTM standard test method D3895. The test conditions are: Uncrimped aluminum pan; no screen; heat up to 200° C. under nitrogen, followed by a switch to a 100 milliliter/minute flow of oxygen. Oxidation induction time (OIT) is the time interval between the start of oxygen flow and the exothermic decomposition of the test specimen. OIT is reported in minutes; under the conditions of this test the longer the OIT the more effective the stabilizer mixture is at delaying the onset of oxidative degradation. Relative performance of stabilizer mixtures in grease filled cable applications can be predicted by comparing the initial OIT values, the aged OIT values and the differences between the initial OIT and aged OIT values.

The stabilizer mixtures containing a metal deactivator and the hindered amines provide excellent performance as measured by initial and aged OIT.

### EXAMPLE 48

Unipol®, Union Carbide Corporation, gas phase polypropylene random copolymer with an initial melt flow rate of ca. 2 dg/min is via addition of a dialkylperoxide, controlled rheology modified to have a target melt flow rate of ca. 25 dg/min, an appropriate melt flow rate for injection molding. A clarifier is added at ca. 2200 ppm to enhance the transparency of the molded articles.

The formulations contain either a binary stabilizer system of the Instant Compounds and an organophosphorus compound, a binary system of the Instant Compounds and one or more compounds selected from the group of hydroxylamine stabilizers, benzofuranone stabilizers and amine oxide stabilizers, or a ternary system of the hindered hydrocarbyloxyamines, one or more compounds selected from the group of hydroxylamine stabilizers, benzofuranone stabilizers and amine oxide stabilizers and an organophosphorus compound.

The Instant Compounds are typically present from about 0.1% to about 1% by weight, the hydroxylamines, benzofuranones and/or amine oxides are typically present from about 0.01% to about 0.5% by weight, and the organic phosphorus compounds are typically present from 0.05% to about 0.5% by weight, based on the overall formulation.

The formulations are prepared by dry blending the appropriate additives with the polymer in a Turbula® blender for twenty minutes followed by melt compounding on a single screw extruder at 500° F. (260° C.) using a polyolefin screw fitted with a Maddock mixing section. Each formulation also contains 750 ppm calcium stearate, 250 ppm of the dialkylperoxide 2,5-bis(tert-butylperoxyl)-2,5-dimethylhexane (90% tech. grade) and 2200 ppm of the Clarifier-1 (Millad® 3988). Each 2 kg batch is split into 1 kg lots, where 1 kg is multiple pass extruded and the other is injection molded into Type IV tensile bars. The Type IV tensile bars, and a set of 125 mil plaques are split into three sets and treated with gamma irradiation from a ⁶⁰Co radiation source at 0, 30 and 60 Kilograys (or 0, 3 and 6 megarads) of exposure. The tensile bars are evaluated for retention of tensile strength and % elongation (at yield, at break) as a function of irradiation dose. The plaques are evaluated for changes in transparency or discoloration as a function of irradiation dose. The irradiated tensile bars, as well as the 125 mil plaques are then oven aged at 60° C. Color and haze development are measured weekly up to 4 weeks on the 125 mil plaques.

A typical organophosphorus stabilizer employed is tris(2,4-di-tert-butylphenyl)phosphite, bis(2,4-di-tert-butylphenyl)pentaerythritol diphosphite, tetrakis(2,4-di-tert-butylphenyl)4,4′-biphenylene-diphosphonite or 2,4,6-tri-t-butylphenyl-(2-ethyl-2-propylpropylidene)phosphite. The amine oxide may be Genox™ EP, a di(C₁₆-C₁₈)alkyl methyl amine oxide, CAS#204933-93-7. The hydroxylamine stabilizer is for example the N,N-di(alkyl)hydroxylamine produced by the direct oxidation of N,N-di(hydrogenated tallow)amine. The benzofuranone stabilizer may be 3-(3,4-dimethylphenyl)-5,7-di-tert-butyl-benzofuran-2-one.

The formulations including the present compounds show superior physical property and color retention.

### EXAMPLE 49

Solution phase Ziegler/Natta high density polyethylene copolymer (d=0.945 g/cm³) with a nominal melt flow rate of ca. 17 dg/min (2.16 kg @ 190° C.) samples are prepared with the additives by adding a 5% additive concentrate to the “additive free” pelleted base resin in a Turbula® blender for twenty minutes followed by melt compounding on a single screw extruder at 450° F. (232° C.) using a polyolefin screw fitted with a Maddock mixing section. The formulations contain the same additives at the levels described in Example 153. Each formulation contains 500 ppm of calcium stearate as an acid scavenger. Each 2 kg batch is split into 1 kg lots and 1 kg is multiple pass extruded and the other is injection molded into Type IV tensile bars or compression molded into 125 mil plaques.

The Type IV tensile bars, 125 mil plaques and 1^{st} pass extrusion pellets are split into three sets and treated with gamma irradiation from a ⁶⁰Co radiation source at 0, 30 and 60 Kilograys (or 0, 3 and 6 megarads) of exposure. The tensile bars are evaluated for retention of tensile strength and % elongation (at yield, at break), the plaques are evaluated for discoloration, and the pellets are tested for retention of melt flow rate, all as a function of irradiation dose. The irradiated tensile bars, as well as the 125 mil plaques, are oven aged at 60° C. Color development, tensile strength and % elongation are measured during oven aging at 60° C.

The formulations containing the Instant Compounds show superior physical property and color retention.

### EXAMPLE 50

Polypropylene homopolymer, Ti/Al catalyst, bulk phase process, with a nominal melt flow index of ca. 15 dg/min at 2.16 kg/230° C. is extruded into fibers at 525° F. and a draw ratio of 3.5:1 and 15 Denier per filament. The fibers are knitted into socks. Samples are also compression molded into plaques. The individual formulations each contain a 1:1 blend of calcium stearate/dihydrotalcite at a total level of 500 ppm as an acid scavenger. Formulations are otherwise prepared as per Example 48.

The fibers, socks and plaques are treated with gamma irradiation from a ⁶⁰Co radiation source at 0, 30, and 60 Kilograys (or 0, 3 and 6 megarads) of exposure.

The formulations containing the present compounds show superior color and/or physical property retention. Such formulations are suitable for woven or nonwoven fibers or filaments or fabrics prepared therefrom.

### EXAMPLE 51

Unipol®, Union Carbide Corporation, gas phase E/H LLDPE copolymer; Ti/Al catalyst; melt index ca. 1 dg/min. at 2.16 kg/190° C. is extruded into blown films at 450° F. to produce 1.5 mil films. The individual formulations each contain zinc stearate at a total level of 500 ppm as an acid scavenger. Formulations are otherwise prepared as per Example 48.

The films are treated with gamma irradiation from a ⁶⁰Co radiation source at 0, 30 and 60 Kilograys (or 0, 3 and 6 megarads) of exposure.

The films containing the formulations of the present invention show superior physical property and color retention.

### EXAMPLE 52

Forming spunbonded fabrics is a conventional process well known in the art. Fiber grade polypropylene is dry blended with 10% loading of the test additive and then melt compounded at 220° C. into masterbatch pellets. The master batch pellets are dry blended with polypropylene resin (MFR=35-50) at a ratio to yield 1.0% additive. Spunbonded fibers are prepared by extrusion of molten polypropylene resin (die temperature=230° C.) as filaments from a plurality of fine circular capillaries of a spinneret. Cooling air is fed into a quenching chamber (2,400 rpm) wherein the filaments are cooled. The cooling air is then sucked through a nozzle, which accelerates the flow of air creating a force that draws the filaments. The drawn filaments are then passed through a diffuser and deposited on a conveyor belt (33 m/min) to form a non-woven fabric.

Forming meltblown fabrics is a conventional process well known in the art. Polypropylene samples are dry blended with 10% loading of a test additive of formulae (1)-(45) respectively, and then melt compounded at 220° C. into masterbatch pellets. The master batch pellets are dry blended with polypropylene resin (MFR 1200) at a ratio to yield 1.0% additive. Meltblown fibers are prepared by extrusion of molten polypropylene resin as filaments from a plurality of fine circular capillaries of a spinneret. A high-velocity heated air stream attenuates the filaments of molten polypropylene to reduce their diameter. There after the meltblown fibers are carried by the high-velocity heated air stream and are deposited on a collection surface to form a web of randomly dispersed meltblown fibers. Thermal bonding of the web to retain integrity and strength occurs as a separate downstream operation.

The nonwoven fabrics containing the present compounds show good UV stability in agricultural applications such as direct covers, small tunnel covers, and shade cloths and also show good stability after exposure to agricultural chemicals such as pesticides and herbicides.

### EXAMPLE 53

### Rotomolding

100 parts medium density polyethylene, copolymerized with hexene (Novapol® TR-0735, nominal melt index 6.8 g/10 min., density 0.935 g/cm³) are dry blended with 0.050 parts of calcium stearate and a combination of additional stabilizers (see below). The mixtures are melt compounded into pellets at 232° C. in a Superior/MPM extruder using a 24:1 L/D screw with Maddock mixing head at 100 rpm.

The compounded pellets are ground to a uniform particle size (150-500 μm) prior to the rotational molding process. This grinding step increases the surface area of the particles leading to a faster heat absorption, and thus reducing overall energy consumption.

The rotational molding process is performed in a laboratory scale equipment FSP M20 “Clamshell”. The ground resin is placed in a cast aluminum mold, which is rotated biaxially in a gas-fired oven. Hot air is circulated by blowers in the chamber while the temperature is increased to 288° C. within 4 minutes. This temperature is maintained for a specific time (see Tables below). Subsequently, the oven is opened and while still rotating, the mold is cooled with forced air circulation for 7 minutes, followed by water spray mist for 7 minutes, and an additional air cooling step for 2 minutes. Throughout the entire heating and cooling cycles; the speed of the major axis is maintained at 6 rpm with a 4.5:1 ratio of rotation. After the cooling cycles, the mold is opened and the hollow object removed.

Formulations are additionally blended with a combination of 0.100 parts of a phosphite process stabilizer, 0.050 parts of a hydroxylamine process stabilizer and 0.200 parts of the instant compounds.

Formulations are additionally blended with a combination of 0.100 parts of a phosphonite process stabilizer, 0.050 parts of a hydroxylamine process stabilizer and 0.200 parts of the instant compounds.

The phosphite stabilizer is tris-(2,4-di-tert-butylphenyl)phosphite or bis(2,4-di-tert-butylphenyl)pentaerythritol diphosphite. The phosphonite stabilizer is tetrakis(2,4-di-tert-butylphenyl)4,4′-biphenylene-diphosphonite. The hydroxylamine stabilizer is the N,N-di(alkyl)hydroxylamine produced by the direct oxidation of N,N-di(hydrogenated tallow)amine.

The formulations are rotationally molded into hollow objects according to the general procedure with hold times of 6 to 14 minutes.

Yellowness Index of the outer surface is determined on a DCI SF600 spectrophotometer according to ASTM D 1925. An increase in yellowness corresponds to a positive increase in the Yellowness Index. The formulations containing the instant compounds exhibit excellent color stability at these processing conditions.

Low-temperature impact strength testing is performed with an instrumented drop weight (11.34 kg/50.8 cm) impact apparatus Dynatup® 8250. Test specimens are conditioned in an air circulated freezer for no less than 12 hours at −40° C. prior to test.

The impact strength results are reported in % brittleness. Formulations containing the instant compounds exhibit excellent stability with regard to the failure mode based on low-temperature impact strength testing at these processing conditions.

Formulations containing the instant compounds also exhibit excellent gas fading resistance. The stabilizer systems described above also exhibit reduced cycle times in the polyolefin rotational molding process compared to the current state of the art systems.

The phosphite bis(2,4-di-tert-butyl-6-methylphenyl)ethyl phosphite may be substituted for the organic phosphorus compounds in the above formulations with excellent results.

Hollow articles are also prepared replacing the hydroxylamine stabilizer used in the formulations with the amine oxide Genox™ EP. Genox™ EP is a di(C₁₆-C₁₈)alkyl methyl amine oxide, CAS#204933-93-7, available from GE Specialty Chemicals. These formulations also exhibit excellent color stability, mechanical stability and improved cycle times.

### EXAMPLE 54

Articles, films and fibers prepared according to the present examples, which are fabricated using recycled plastics as part of the formulation and the stabilizers of the present invention, exhibit stability against the deleterious effects of actinic light and thermal exposure.

### EXAMPLE 55

All additives are commercial materials. Titanium dioxide is DuPont Ti-PURE® R-104. Polycarbonate (PC) is LEXAN® 141-111 natural; ABS is Dow MAGNUM® 342EZ. Polymers and additives are extrusion compounded in one pass using a twin screw extruder of screw design 18 mm, co-rotating, non-intermeshing; processing temperature 240° C., with a die melt temperature 260° C. Injection molding of Izod bars (2.5″ L×0.5″ W×0.125″ W) is done on a BOY 30 machine, barrel temperature 475-515° F., die temperature 515° F.

Accelerated weathering is performed using an Atlas Ci65A Weather-Ometer (“XAW”), operated in either “Dry XAW” mode (ASTM G26-90 method C) or interior auto (“IAXAW”) (black panel temperature 89° C., light/dark cycle 3.8 hr/1.0 hr; irradiance 0.55 watt/m², inner filter-quartz, outer filter-high borate.

Yellowness Index and delta E color are performed as per ASTM D-1925, using a Chroma-Sensor CS-5 Colorimeter, operated at 10 degree, small area view, specular included.

Chip impact is performed per ASTM D4508-90 on a TMI Monitor/Impact Tester using a 30 ft-lb weight. Per ASTM D4508-90 paragraph 4.6, the impact strength results are reported as a complete break, a partial break or a non-break, with a non-break specimen exhibiting the most desired impact retention, and partial break being the next desired impact retention. Data presented are the average of ten replicate samples for each formulation.

Melt rheology is performed using a Kayeness Galaxy V capillary rheometer, equipped with a 1000 pound load cell, a die of orifice radius 0.015 inch and orifice length 1.0000 inch., operated at the stated test temperature. Samples are pre-dried in a vacuum oven under nitrogen atmosphere to an analyzed moisture level of less than 100 ppm prior to rheology testing.

The stabilizers used in the tests below are:

UVA: 2-(2-hydroxy-3,5-di-alplha-cumylphenyl)-2H-benzotriazole or 4,6-diphenyl-2-(4-hexyloxy-2-hydroxyphenyl)-s-triazine

Phosphite is tris(2,4-di-tert-butylphenyl)phosphite.

Chip impact bars are prepared and subjected to dry XAW accelerated weathering. The samples are removed at selected intervals for color measurement and then for destructive chip impact testing.

Formulations containing 0.75 weight percent of the present compounds and 0.5 weight percent of UVA exhibit outstanding color stability and retention of useful mechanical properties as measured by chip impact strength.

All formulations contain 0.1% Phosphite; all are 50/50 wt/wt PC and ABS.

### EXAMPLE 56

Chip impact bars are prepared as per Example 55 and subjected to accelerated weathering under interior auto Xenon Arc Weather-Ometer (IAXAW) conditions. Test samples are removed at intervals for color measurement and then destructive chip impact testing. The IAXAW conditions are more severe than dry Xenon (XAW) conditions due to higher temperature and higher irradiance energy.

Formulations containing 0.75 weight percent of the present compounds in addition to 0.5 weight percent of UVA exhibit outstanding color stability and retention of useful mechanical properties as measured by chip impact strength.

All formulations contain (0.1%) tris(2,4-di-tert-butylphenyl)phosphite; all are 50/50 wt/wt PC and ABS.

### EXAMPLE 57

Formulations of a 50/50 wt/wt blend of PC and ABS which contain 1.0 wt. % titanium dioxide (TiO₂), 0.75 weight percent of the present compounds and 0.50 weight percent of either 2-(2-hydroxy-3,5-di-alplha-cumylphenyl)-2H-benzotriazole or 4,6-diphenyl-2-(4-hexyloxy-2-hydroxyphenyl)-s-triazine are prepared and tested as per Example 55. The formulations exhibit outstanding resistance to color change (delta E). The chip impact values are favorably high as well.

All formulations contain (0.1%) tris(2,4-di-tert-butylphenyl)phosphite; all are 50/50 wt/wt PC and ABS.

### EXAMPLE 58

The retention of molecular weight during processing of a polymer or polymer blend is critical for the successful production of a useful article. Additives that interact adversely with the polymer substrate during processing are of limited or no value to the production of a final useful article. The adverse interactions may be due to various factors, such as a chemical reaction between the additive and the polymer that results in a reduction of polymer molecular weight by chain cleavage. Also, if the additive itself degrades during the required high temperature processing of the polymer, then the degradation products of the additive itself may interact antagonistically with the polymer.

An acceptable hindered amine stabilizer for use in a polycarbonate blend should exhibit minimal deleterious interaction with the polymers during their high temperature processing into useful articles. Hindered amines that are basic (having a pKa value >7 typically) are known to cause degradation of polycarbonates, especially during melt processing of the polymer as taught by G. L. Gaines, Jr. (Polymer Degradation and Stability, 27 13-18 (1990)). Melt rheology testing is an accepted method to assess the stability of a polymer in the melt state (see ASTM D 3835-90) and thus to relate the interaction of additives to changes in molecular weight of the polymers. Polymer molecular weight, and changes in molecular weight, may be expressed in terms of the polymer apparent melt viscosity. A decrease in apparent melt viscosity over time of the test indicates that polymer degradation and molecular weight reduction are occurring whereas an increase in apparent melt viscosity over time indicates polymer degradation may be occurring by a crosslinking or a molecular weight buildup mechanism. It is often convenient to represent this change in melt viscosity as the melt viscosity ratio as taught by A. B. Auerbach et al., Polymer Engineering and Science, 30, 1041-1050 (1990). The melt viscosity ratio (MVR) may be defined as the change in melt viscosity (η) over time, and expressed as the ratio of a melt viscosity at some extended test time (ηₓ), divided by the initial melt viscosity (ηₒ) ultimately expressed as (ηₓ/ηₒ).

Formulations of 50/50 wt/wt PC/ABS samples containing the present hindered amines, alone and polymer samples additionally containing various additives that are incorporated as described for Examples 55 and 56 exhibit low detrimental impact on apparent melt viscosity of formulations.

The PC/ABS blends of Examples 55-57 may be replaced with PC/ASA blends which also exhibit excellent color stability and mechanical stability as measured by impact strength and melt flow stability.

The PC/ABS blends of Examples 55-57 may be replaced with PC/polyester blends such as PC/PET and PC/PBT blends which also exhibit excellent color stability and mechanical stability as measured by impact strength and melt flow stability.

ASA is acrylonitrile-styrene-acrylate, the acrylate is typically butyl acrylate. PET is polyethylene terephthalate. PBT is polybutylene terephthalate. ABS is acrylonitrile-butadiene-styrene.

### EXAMPLE 59

### Coextrusion Over PVC

A sheet composition suitable for use in weatherable house siding is prepared by coextrusion of a 0.010″ thick light stabilized rigid PVC layer (“cap layer”) over a 0.060″ thick rigid PVC bulk substrate (“bulk layer”). Composition of the layers is given below.

| | | |
|---|---|---|
| Cap Layer | 100.00 | phr PVC |
| | 2.50 | phr IRGASTAB ® T 634 (thermal stabilizer) |
| | 6.00 | phr KM-334 (acrylic impact modifier - |
| | | Rohm & Haas) |
| | 1.50 | phr K-120N (processing aid - Rohm & Haas) |
| | 0.60 | phr Paraffin Wax 165 (Rheolube) |
| | 0.30 | phr PE Wax AC629A (Honeywell) |
| | 0.50 | phr Gray color concentrate |
| | 4.70 | phr R-960 titanium dioxide (DuPont) |
| | 0.50 | phr Instant Compound |
| | 0.50 | phr BZT UV absorber |
| Bulk Layer | 100.00 | phr PVC |
| | 2.50 | phr IRGASTAB ® T 634 (thermal stabilizer) |
| | 6.00 | phr KM-334 (acrylic impact modifier - |
| | | Rohm & Haas) |
| | 1.50 | phr K-120N (processing aid - Rohm & Haas) |
| | 0.60 | phr Paraffin Wax 165 (Rheolube) |
| | 0.30 | phr PE Wax AC629A (Honeywell) |

The weatherability of the sheets of the present invention, containing the instant hindered amines, respectively, is superior to a control sheet prepared without the use of the instant compounds in the cap layer.

BZT UV absorber is 2-(3,5-di-t-amyl-2-hydroxyphenyl)-2H-benzotriazole.

The PVC cap layer may be replaced with ASA, PMMA, polyvinylidene fluoride (PVDF) or polypropylene-PMMA graft copolymer (PP-g-PMMA) cap layers as per the following formulations. In each case the bulk layer is as above.

| | | |
|---|---|---|
| Cap Layer | 100.00 | phr ASA (e.g. GELOY ® from GE Plastics) |
| | 0.50 | phr Gray color concentrate |
| | 4.70 | phr R-960 titanium dioxide (DuPont) |
| | 0.50 | phr Instant Compound |
| | 0.50 | phr BZT UV absorber A |
| | 0.10 | phr process stabilizer A |
| Cap Layer | 100.00 | phr impact-modified PMMA |
| | 0.50 | phr Gray color concentrate |
| | 4.70 | phr R-960 titanium dioxide (DuPont) |
| | 0.50 | phr Instant Compound |
| | 0.50 | phr BZT UV absorber B |
| | 0.10 | phr process stabilizer A |
| Cap Layer | 100.00 | phr PVDF (e.g. KYNAR ® from Elf Atochem) |
| | 0.50 | phr Gray color concentrate |
| | 4.70 | phr R-960 titanium dioxide (DuPont) |
| | 0.50 | phr Instant Compound |
| | 0.50 | phr BZT UV absorber C |
| Cap Layer | 100.00 | phr PP-g-PMMA (e.g. INTERLOY ® from |
| | | Montell) |
| | 0.50 | phr Gray color concentrate |
| | 4.70 | phr R-960 titanium dioxide (DuPont) |
| | 0.20 | phr Instant Compound |
| | 0.20 | phr oligomeric HALS |
| | 0.20 | phr BZT UV absorber A |
| | 0.10 | phr process stabilizer B |

In each case, the weatherability of the sheets of the present invention, containing the instant compounds is superior to a control sheet prepared without the use of the instant compounds in the cap layer.

BZT UV absorber A is 2-(3,5-di-t-amyl-2-hydroxyphenyl)-2H-benzotriazole; BZT UV absorber B is 2-(2-hydroxy-3,5-di-alpha-cumylphenyl)-2H-benzotriazole; BZT UV absorber C is 2-(3-t-butyl-2-hydroxy-5-(2-(ω-hydroxy-octa-(ethyleneoxy)carbonyl-ethyl)-, phenyl)-2H-benzotriazole. Oligomeric HALS is N-{[2-(N-2,2,6,6-tetramethylpiperidin-4-yl)butylamino]-s-triazin-4-yl)-N,N′-bis(2,2,6,6-tetramethylpiperidin-4-yl)-1,6-hexanediamine terminated with 2,4-bis(dibutylamino)-s-triazin-6-yl. Process stabilizer A is a 80:20 blend of tris(2,4-di-tert-butylphenyl)phosphite and β-(3,5-di-tert-butyl-4-hydroxyphenyl)propionic acid octadecanoic ester. Process stabilizer B is a 1:1 blend of tris(2,4-di-tert-butylphenyl)phosphite and the N,N-di(alkyl)hydroxylamine produced by the direct oxidation of N,N-di(hydrogenated tallow)amine.

### EXAMPLE 60

### Coextrusion Over Polycarbonate

A sheet composition suitable for use in weatherable glazing is prepared by coextrusion of a 0.010″ thick light stabilized PMMA layer (“cap layer”) over a 0.100″ thick polycarbonate bulk substrate (“bulk layer”). Composition of the layers is given in the table below.

| | | |
|---|---|---|
| Cap Layer | 100.00 | phr PMMA |
| | 0.10 | phr process stabilizer |
| | 0.25 | phr Instant Compound |
| | 3.50 | phr HPT UV absorber |
| Bulk Layer | 100.00 | phr Polycarbonate (e.g. LEXAN ® 141 from GE) |
| | 0.08 | phr phosphite process stabilizer |
| | 0.10 | phr BZT UV absorber |

The weatherability of the sheets of the present invention, containing the instant compounds is superior to a control sheet prepared without the use of the instant compounds in the cap layer.

Coextruded sheets are also prepared, replacing HPT UV absorber in the cap layer with each of 2,2′-methylene-bis(4-t-octyl-(6-2H-benzotriazol-2-yl)phenol) and 2-(2-hydroxy-3,5-di-alpha-cumylphenyl)-2H-benzotriazole. Excellent results are achieved for these coextruded sheets containing the instant hindered amines.

BZT UV absorber is 2-(2-hydroxy-3,5-di-alpha-cumylphenyl)-2H-benzotriazole, HPT UV absorber is 4,6-diphenyl-2-(4-hexyloxy-2-hydroxyphenyl)-s-triazine. Phosphite stabilizer is tris(2,4-di-tert-butylphenyl)phosphite. Process stabilizer is a 80:20 blend of tris(2,4-di-tert-butylphenyl)phosphite and β-(3,5-di-tert-butyl-4-hydroxyphenyl)propionic acid octadecanoic ester.

### EXAMPLE 61

### Coextrusion

A composition suitable for use as weatherable window profile is prepared by coextrusion of a 0.010″ thick light stabilized ASA layer (“cap layer”) over a 0.060″ thick ABS bulk substrate (“bulk layer”). Composition of the layers is given in the table below.

| | | |
|---|---|---|
| Cap Layer | 100.00 | phr ASA (e.g. GELOY ® from GE Plastics) |
| | 4.00 | phr R-960 titanium dioxide (DuPont) |
| | 0.50 | phr Instant Compound |
| | 0.50 | phr BZT UV absorber |
| | 0.10 | phr process stabilizer |
| Bulk Layer | 100.00 | phr ABS (e.g. CYCOLAC ® from GE Plastics) |
| | 0.10 | phr process stabilizer |

The weatherability of the sheets of the present invention, containing the instant compounds, is superior to a control sheet prepared without the use of the instant compounds in the cap layer.

BZT UV absorber is 2-(2-hydroxy-3,5-di-alpha-cumylphenyl)-2H-benzotriazole. Process stabilizer is a 80:20 blend of tris(2,4-di-tert-butylphenyl)phosphite and β-(3,5-di-tert-butyl-4-hydroxyphenyl)propionic acid octadecanoic ester.

### EXAMPLE 62

### Multilayer Polymer Structures

The instant compounds have utility as light stabilizers to protect light sensitive polymers present in multilayer polymer structures. Examples of such polymer structures include but are not limited to:
1.) Sheets and signs as seen in WO97/42261; and U.S. Pat. No. 5,387,458 which are incorporated herein by reference;
2.) Solar Control Films of Various Construction as seen in U.S. Pat. Nos. 3,290,203, 3,681,179, 3,776,805 and 4,095,013, incorporated herein by reference; and
3.) Base stock or cap stock for coextrusion structures such as window profiles, laminates over automotive bumpers or auto exterior panels.

Window profiles include photosensitive polymers such as ABS, ASA, SAN or vinylogous polymers such as PVC. Automotive polymeric materials which are photosensitive include for example ABS, SAN, ASA and polycarbonate as well as blends such as PC/ABS, which include Pulse® from Dow, Cycoloy® from GE, Bayblend® from Bayer, PC/PBT known as Xenoy® from GE, PC/ASA such as Geloy® from GE, and the “W4” polymer as disclosed by General Electric Company (Modern Plastics May 2000 pages 90-91).

The instant compounds of this disclosure act to protect against photolytic degradation of a polymer component, or an incorporated pigment, dye colorant, or protect adhesive or “tie-layers” in such constructions.

A multilayer polymer composite is prepared by different routes, such as co-extrusion of one or more polymer compositions to form the multilayer composite. Alternatively, compression molding or thermoforming of one or polymer compositions produces the desired polymer composite. In particular, these techniques are used in the manufacture of signage, typically composed of one or more layers of polymeric materials formed on top of a base material (metal sheet, plastic, etc).

Examples of potential polymeric materials that may comprise one or more sections of the laminate, sign, sheet-or composite structure may include:
polycarbonate
polyesters such as PET, PBT, PEN, PTT
acrylics such as PMMA and acrylate copolymer or terpolymers
polyolefins
vinylogous polymers and copolymers composed of vinyl chloride, vinyl acetate,
vinylidene chloride, vinylidene fluoride.

The present compounds each provide excellent stabilization to such constructions.

### EXAMPLE 63

### Photo-Cured White Screen Ink

A model white UV-curable screen ink, based on urethane acrylate chemistry, is prepared with a fixed pigment to binder ratio of 0.74. A base formulation is prepared which is complete excluding a photoinitiator and a hindered amine of component (b) and consists of:
Rutile TiO₂, 500.0 g
Ebecryl® 284, 300.0 g
Ebecryl® 810, 100.0 g
Trimethylolpropane ethoxy triacrylate (TMPEOTA), 60.0 g
Tripropylene glycol diacrylate (TRPGDA), 180.0 g
Isobornyl acrylate (IBOA), 30.0 g
Modaflow® 2100, 5.0 g
Byk®-A 501, 5.0 g
Aerosil® 200, 20.0 g

The TiO₂ is added as a 66% dispersion in a portion of the Ebecryl® 284. The Aerosil® 200 is added as a 10% dispersion in the TRPGDA.

Ebecryl® 284 is an acrylated aliphatic urethane oligomer/monomer blend. Ebecryl® 810 is a polyester acrylate oligomer. The Ebecryl® products as well as the acrylate monomers are available from UCB Chemicals Corp., Smyrna, Ga. Modaflow® 2100 is an acrylic flow agent available from Monsanto. Byk®-A 501 is an air release agent available from Byk-Chemie. Aerosil® 200 is a fumed silica viscosity modifier from Degussa.

To a portion of the base formulation is added a photoinitiator mixture of bis(2,4,6-trimethylbenzoyl)phenylphosphine oxide/1-hydroxycyclohexylphenylketone in a 35/65 ratio. The photoinitiator mixture is 4.78 weight percent of the formulation.

The formulations further contain 1% each of the instant hindered amines of the present disclosure.

Eight replicate prints of each formulation are prepared. Application is made via a 355 mesh screen and applied on polycarbonate sheets. Samples are cured with a moving belt at 50 feet/min under two medium pressure mercury lamps perpendicular to the belts @ 200 watts/in. each. All prints receive two passes under the lamps. Final layer thickness is approximately 1.2 to 1.3 mils (31-34 microns).

The present hindered amines provide excellent stabilization to the photo-cured inks.

### EXAMPLE 64

### Photo-Cured White Pigmented Coating

A model white UV-curable coating for wood is prepared based on polyester acrylate chemistry with a titanium dioxide level of 25% by weight. A base formulation is prepared containing:
Rutile TiO₂, 100.0 g
Ebercryl® 830, 240.0 g
HDODA, 42 g
TMPTA, 18.0 g

The TiO₂ is added as a 63% dispersion in a portion of the Ebercryl® 830. Ebercryl® 830 is a hexafunctional polyester acrylate oligomer. HDODA is 1,6-hexanediol diacrylate. TMPTA is trimethylolpropane triacrylate. The Ebecryl® products as well as the acrylate monomers are available from UCB Chemicals Corp., Smyrna, Ga.

To a portion of the base formulation is added a photoinitiator mixture of bis(2,4,6-trimethylbenzoyl)phenylphosphine oxide/1-hydroxycyclohexylphenylketone in a 1:2 ratio. The photoinitiator mixture is 3.0 weight percent of the total formulation.

The formulations further contain 1% each of the instant compounds of the present disclosure.

Eight replicate prints of each formulation are prepared. Films are prepared with a draw-down bar over a white Scotchcal® vinyl film from 3M. Samples are cured with a moving belt at 58 feet/min. under two medium pressure mercury lamps perpendicular to the belts @ 300 watts/in. each. The prints received one pass under the lamps. Irradiance received is 618 mJ/cm². Final cured thickness is 2.1 mils (53 microns).

The present compounds provide excellent stabilization to the photo-cured coatings.

### EXAMPLE 65

### Photo-Cured Clear Coating

A model clear UV-curable coating for wood is prepared based on acrylated aromatic urethane/epoxy chemistry. A base formulation is prepared comprising:
Ebercryl® 4827, 30.0 g
Ebercryl® 600, 30.0 g
Tripropylene glycol diacrylate (TRPGDA), 40.0 g

Ebercryl® 4827 is an aromatic urethane diacrylate oligomer. Ebercryl® 600 is the diacrylate ester of a bisphenol-A epoxy resin. The Ebecryl® products as well as the acrylate monomers are available from UCB Chemicals Corp., Smyrna, Ga.

To a portion of the base formulation is added a photoinitiator mixture of bis(2,4,6-trimethylbenzoyl)phenylphosphine oxide/1-hydroxycyclohexylphenylketone in a 1:2 ratio. The photoinitiator mixture is 3.0 weight percent of the formulation.

The formulations further contain 1% each of the instant compounds of the present disclosure.

Four replicate prints of each formulation are prepared. Films are prepared with a draw-down bar over a white Scotchcal® vinyl film from 3M. Samples are cured with a moving belt at 95. feet/min. under two medium pressure mercury lamps perpendicular to the belts @ 300 watts/in. each. The prints received two passes under the lamps. Irradiance received is 750 mJ/cm². Final cured thickness is 5.1 mils (130 microns).

The coatings containing in addition to a bisacylphosphine oxide photoinitiator, a hindered amine of the instant invention display excellent stability.

What is claimed is:

1. A composition comprising
(a) candle wax which is white and unscented; white and scented; dyed and unscented; dyed and scented; dipped and unscented; or dipped and scented, and
(b) an effective stabilizing amount of a combination of
(i) a substituted hexahydro-1,4-diazepin-5-one compound which compound comprises one or more a moiety of formula (I) and 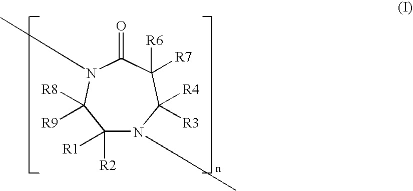
(ii) a UV absorber or an antioxidant, or a UV absorber and an antioxidant
wherein the ratio by weight of (i) to (ii) is from about 10:1 to about 1:10;
wherein
n is 1 or 2,
R₁, R₂, R₃ and R₄ are independently alkyl of 1 to 12 of carbon atoms, said alkyl substituted by one or two hydroxyl groups; or R₁ and R₂ together or R₃ and R₄ together or R₁ and R₂ together and R₃ and R₄ together are tetramethylene, pentamethylene or pentamethylene substituted by methyl;
R₆, R₇, R₈ and R₉ are independently hydrogen, alkyl of 1 to 12 of carbon atoms, said alkyl substituted by one or two hydroxyl groups; or R₆ and R₇ together or R₈ and R₉ together or R₆ and R₇ together and R₈ and R₉ together are tetramethylene, pentamethylene or pentamethylene substituted by methyl.

2. A composition according to claim 1 wherein the substituted hexahydro-1,4-diazepin-5-one compound of component (b)(i) is a compound of formula (II), (III), or (IV) 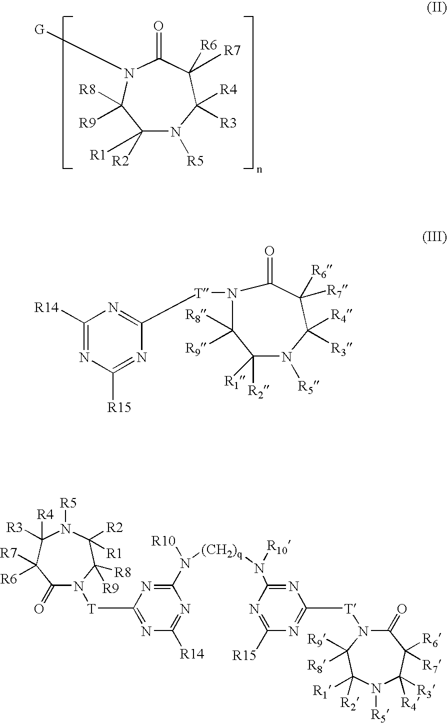
wherein
n is 1 or 2,
p is 1 to 12,
when n is 1,
G is hydrogen, alkyl of 1 to 18 carbon atoms, alkyl of 2 to 18 carbon atoms substituted by NH₂, by OH, by halogen, by —OR₁₀, by 4-hydroxy-3,5-di-tert-butylphenyl or by 4-hydroxy-3,5-dimethyl-phenyl, or by a mixture of these groups; or G is alkyl of 2 to 18 carbon atoms interrupted by —O—, by —CO—, by —NH— or by —NR₁₀— or by a mixture of said groups; or G is alkyl of 1 to 18 carbon atoms substituted by —COOR₁₁ or by phenyl;
when n is 2,
G is alkylene of 2 to 10 carbon atoms, alkenylene of 4 to 12 carbon atoms, alkylene of 3 to 15 carbon atoms substituted by one to four OH or said alkylene interrupted by one to four oxygen atoms; or G is phenylene, C₁-C₄alkylphenylene, —CO—(CH₂)ₚ—CO—, or —COO—;
R₁, R₂, R₃ and R₄ are independently alkyl of 1 to 12 of carbon atoms, said alkyl substituted by one or two hydroxyl groups; or R₁ and R₂ together or R₃ and R₄ together or R₁ and R₂ together and R₃ and R₄ together are tetramethylene, pentamethylene or pentamethylene substituted by methyl;
R₁′, R₂′, R₃′ and R₄′ and R₁″, R₂″, R₃″ and R₄″ are defined independently as for R₁, R₂, R₃ and R₄;
R₆, R₇, R₈ and R₉ are independently hydrogen, alkyl of 1 to 12 of carbon atoms, said alkyl substituted by one or two hydroxyl groups; or R₆ and R₇ together or R₈ and R₉ together or R₆ and R₇ together and R₈ and R₉ together are tetramethylene, pentamethylene or pentamethylene substituted by methyl;
R₆′, R₇′, R₈′ and R₈′ and R₆″, R₇″, R₈″ and R₉″ are defined independently as for R₆, R₇, R₈, and R₉;
R₅ is hydrogen, oxyl, hydroxyl, cyanoethyl, phenylalkyl of 7 to 15 carbon atoms, said phenylalkyl substituted on the phenyl ring by one to three radicals selected from alkyl of 1 to 4 carbon atoms and alkoxy of 1 to 4 carbon atoms, phenylalkoxy of 7 to 15 carbon atoms, said phenylalkoxy substituted on the phenyl ring by one to three radicals selected from alkyl of 1 to 4 carbon atoms and alkoxy of 1 to 4 carbon atoms, cycloalkoxy of 5 to 12 carbon atoms, alkenyl of 3 to 8 carbon atoms, alkynyl of 3 to 8 carbon atoms, alkoxy of 1 to 24 carbon atoms, alkyl of 1 to 24 carbon atoms, said alkyl substituted by one or two hydroxyl groups, said alkyl substituted by alkylcarbonyloxy of 2 to 18 carbon atoms, or said alkyl interrupted by one to ten oxygen atoms and substituted by one hydroxyl, by alkylcarbonyloxy of 2 to 18 carbon atoms or by 4-hydroxy-3,5-di-tert-butylbenzoyloxy; or R₅ is alkanoyl of 1 to 8 carbon atoms, alkenoyl of 3 to 5 carbon atoms, alkanoyloxy of 1 to 18 carbon atoms, glycidyl, or a group —CH₂ CH(OH)-E where E is hydrogen, methyl or phenyl;
T, T′, and T″ are independently defined as alkylene of 1 to 12 carbon atoms; said alkylene interrupted by one to four oxygen atoms; said alkylene interrupted by one to four —NH— groups or —N(R₁₀)— groups; said alkylene interrupted by —OC(O)—, —C(O)—, —C(O)N(H)—, or —C(O)N(R₁₀)—; said alkylene substituted by one to four hydroxyl groups; said alkylene substituted by one to four —NH₂ groups or —N(R₁₀)H groups or —N(R₁₀)(R₁₃) groups; or T, T′, and T″ are independently —CH₂—CH(R₁₂)—O— wherein R₁₂ is hydrogen, methyl or phenyl, or T, T′, and T″ are independently —(CH₂)₃—NH— or T, T′, and T″ are independently a direct bond;
R₅′ and R₅″ are defined as for R₅;
R₁₀ and R₁₀′ are independently alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;
R₁₃ is alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;
R₆′ and R₆″ are defined as for R₆;
R₁₁ and R₁₁′ are independently hydrogen, alkyl of 1 to 18 carbon atoms, alkyl of 2 to 18 carbon atoms which is substituted by —NH₂, by —NHR₁₀, by —N(R₁₀)₂, by nitro, by hydroxy, by alkoxy of 1 to 18 carbon atoms or by a mixture of said groups; or R₁₁ and R₁₁′ are alkenyl of 3 to 18 carbon atoms, cycloalkyl of 5 to 12 carbon atoms, or said cycloalkyl which is substituted by alkyl of 1 to 4 carbon atoms or is interrupted by —O—, or R₁₁ and R₁₁′ is phenyl or phenylalkyl of 7 to 15 carbon atoms;
R₁₄ is —N(R₁₆)(R₁₆′) or is chlorine, alkoxy of 1 to 12 carbon atoms, thioalkyl of one to eighteen carbon atoms, 2-hydroxyethylamino or —N(R₁₁)(R₁₁′);
or R₁₄ is 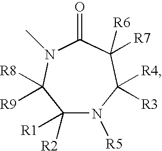
R₁₅ is defined as for R₁₄;
R₁₆ and R₁₆′ are independently glycidyl, C₂-C₁₂alkanoyl substituted by a di(C₁-C₆alkyl) phosphonate, or
R₁₆ and R₁₆′ are independently C₁-C₁₂alkyl, C₂-C₁₂alkanoyl or C₇-C₁₈phenylalkyl, each interrupted by one to six oxygen, sulfur or —N(R₁₀)— groups; C₁-C₁₂alkyl, C₂-C₁₂alkanoyl, phenyl or C₇-C₁₈phenylalkyl, each substituted by one to six hydroxy groups or by one to six —NHR₁₀ groups; C₂-C₁₂alkyl, C₂-C₁₂alkanoyl or C₇-C₁₈phenylalkyl, each interrupted by one to three —NR₁₀ C(O)— groups; or R₁₆ and R₁₆′ are independently C₁-C₁₂alkyl, C₂-C₁₂alkanoyl, phenyl or C₇-C₁₈phenylalkyl, each substituted by one to three —SO₃H groups or by one to three —COOR₁₁ groups; and
q is 2 to 8.

3. A composition according to claim 2 where in the hexahydro-1,4-diazepin-5-one compound of formula (II), (III), or (IV), R₁, R₂, R₃ and R₄, R₁′, R₂′, R₃′ and R₄′ and R₁″, R₂″, R₃″ and R₄″ are defined independently and are each methyl, or R₁ and R₂ together and R₃ and R₄ together are pentamethylene.

4. A composition according to claim 2 where in the hexahydro-1,4-diazepin-5-one compound of formula (II), (III), or (IV), R₅, R₅′ and R₅″ are hydrogen or methyl.

5. A composition according to claim 2 wherein the hexahydro-1,4-diazepin-5-one compound of formula (II), (III), and (IV) of component (b) (i) is
(a) 1,3,5-tris(1-cyclohexyl-2,2,7,7-tetramethylhexahydro1,4-diazepin-5-on-4-yl)-s-triazine;
(b) 1,3,5-tris(1,2,2,7,7-pentamethylhexahydro1,4-diazepin-5-on-4-yl)-s-triazine;
(c) 1,2,2,7,7-pentamethylhexahydro-1,4-diazepin-5-one;
(d) 1,2-ethane-bis(1,2,2,7,7-pentamethylhexahydro1,4-diazepin-5-on-4-yl);
(e) 3,3,5,5-tetramethyl-7-oxo-[1,4]diazepine-1-carboxylic acid ethyl ester;
(f) 3,3,5,5-tetramethyl-7-oxo-[1,4]diazepine-1-carboxylic acid methyl amide;
(g) 3,3,5,5-tetramethyl-7-oxo-[1,4]diazepine-1-carboxylic acid hexyl amide;
(h) 4-benzyl-2,2,7,7-tetramethylhexahydro-1,4-diazepin-5-one;
(i) 1-hydroxyl-4-benzyl-2,2,7,7-tetramethylhexahydro-1,4-diazepin-5-one;
(j) 2,7-diethyl-1,2,3,7-tetramethylhexahydro-1,4-diazepin-5-one;
(k) 1-octyloxy-4-benzyl-2,2,7,7-tetramethylhexahydro-1,4-diazepin-5-one;
(l) 1-acetyl-2,2,7,7-tetramethylhexahydro-1,4-diazepin-5-one;
(m) 1-oxyl-4-benzyl-2,2,7,7-tetramethylhexahydro-1,4-diazepin-5-one;
(n) 1,3-bis(1-cyclohexyloxy-2,2,7,7-tetramethylhexahydro1,4-diazepin-5-on-4-yl)-5-(2-hydroxylethyl)amino-s-triazine;
(o) 1-(1-octyloxy-2,2,7,7-tetramethylhexahydro1,4-diazepin-5-on-4-yl)-3,5-bis(2-hydroxylethyl)amino-s-triazine;
(p) 1-(1-methoxy-2,2,7,7-tetramethylhexahydro1,4-diazepin-5-on-4-yl)-3,5-bis(methylthio)-s-triazine; or
(q) 1,3-bis(1-cyclohexyl-2,2,7,7-tetramethylhexahydrol1,4-diazepin-5-on-4-yl)-5-(2-hydroxylethyl)amino-s-triazine.

6. A composition according to claim 5 wherein the hexahydro-1,4-diazepin-5-one compound of formula (II), (III), and (IV) of component (b)(i) is
(a) 1,3,5-tris(1-cyclohexyl-2,2,7,7-tetramethylhexahydro1,4-diazepin-5-on-4-yl)-s-triazine;
(b) 1,3,5-tris(1,2,2,7,7-pentamethylhexahydro1,4-diazepin-5-on-4-yl)-s-triazine;
(c) 1,3-bis(1-cyclohexyloxy-2,2,7,7-tetramethylhexahydro1,4-diazepin-5-on-4-yl)-5-(2-hydroxylethyl)amino-s-triazine;
(d) 1,3-bis(1-cyclohexyl-2,2,7,7-tetramethylhexahydro1,4-diazepin-5-on-4-yl)-5-(2-hydroxylethyl)amino-s-triazine;
(e) 1-(1-octyloxy-2,2,7,7-tetramethylhexahydro1,4-diazepin-5-on-4-yl)-3,5-bis(2-hydroxylethyl)amino-s-triazine; or
(f) 1-(1-methoxy-2,2,7,7-tetramethylhexahydro1,4-diazepin-5-on-4-yl)-3,5-bis(methylthio)-s-triazine.

7. A composition according to claim 1 wherein the ratio by weight of (i) to (ii) is from about 4:1 to about 1:4.

8. A composition according to claim 7 wherein the ratio by weight of (i) to (ii) is from about 2:1 to about 1:2.

9. A composition according to claim 1 wherein the effective amount of the UV absorber plus the hexahydro-1,4-diazepin-5-one compound in the candle wax is from about 0.01 to about 10% by weight based on the wax.

10. A composition according to claim 9 wherein the effective amount of the UV absorber plus the hexahydro-1,4-diazepin-5-one compound in the candle wax is from about 0.1 to about 2% by weight based on the wax.

11. A composition according to claim 10 wherein the effective amount of the UV absorber plus the hexahydro-1,4-diazepin-5-one compound in the candle wax is from about 0.1 to about 0.5% by weight based on the wax.

12. A composition according to claim 1 wherein the UV absorber of component (ii) is a benzotriazole, a benzophenone, an α-cyanoacrylate, an oxanilide, an s-triazine, a cinnamate, a malonate, a benzoate or a salicylate, or a mixture thereof.

13. A composition according to claim 12 wherein the UV absorber is a benzotriazole, a benzophenone or an s-triazine.

14. A composition according to claim 13 wherein the UV absorber is
(a) 4-octyloxy-2-hydroxybenzophenone;
(b) 4-methoxy-2-hydroxybenzophenone;
(c) 2-(2-hydroxy-5-methylphenyl)-2H-benzotriazole;
(d) 2-(2-hydroxy-5-tert-octylphenyl-2H-benzotriazole;
(e) 2-(2-hydroxy-3,5-di-tert-amylphenyl)-2H-benzotriazole;
(f) octyl 3-(benzotriazol-2-yl)-5-tert-butyl-4-hydroxyhydrocinnamate;
(g) 2-(2-hydroxy-3,5-di-tert-butylphenyl)-2H-benzotriazole;
(h) 2-(2-hydroxy-5-tert-butylphenyl)-2H-benzotriazole;
(i) 2-(2-hydroxy-3,5-di-tert-butylphenyl)-2H-benzotriazole;
(j) 2-(2-hydroxy-3-tert-butyl-5-methylphenyl)-2H-benzotriazole;
(k) 2-(2-hydroxy-3-sec-butyl-5-tert-butylphenyl)-2H-benzotriazole;
(l) 2-(2-hydroxy-4-octyloxyphenyl)-2H-benzotriazole;
(m) 2-(2-hydroxy-3-dodecyl-5-methylphenyl)-2H-benzotriazole;
(n) 2-[2-hydroxy-3,5-di(α,α-dimethylbenzyl)phenyl]-2H-benzotriazole;
(o) 2-[2-hydroxy-3-(α,α-dimethylbenzyl)-5-tert-octylphenyl]-2H-benzotriazole;
(p) 2-{2-hydroxy-3-tert-butyl-5-[2-(omega-hydroxy-octa(ethyleneoxy)carbonyl)ethyl]-phenyl}-2H-benzotriazole;
(q) 2-{2-hydroxy-3-tert-butyl-5-[2-(octyloxy)carbonyl)ethyl]phenyl}-2H-benzotriazole;
(r) 2-ethylhexyl p-methoxycinnamate;
(s) 4-methoxy-2,2′-dihydroxybenzophenone;
(t) 4,4′dimethoxy-2,2′-dihydroxybenzophenone;
(u) 2,4-bis(2,4-dimethylphenyl)-6-(2-hydroxy-4-octyloxyphenyl)-s-triazine;
(v) 2,4-diphenyl-6-(2-hydroxy-4-hexyloxyphenyl)-s-triazine;
(w) 2,4-bis(2,4-dimethylphenyl)-6-[2-hydroxy-4-(3-do-/tri-decyloxy-2-hydroxypropoxy)-phenyl]-s-triazine; (x) 2,4-bis(2,4-dimethylphenyl)-6-[2-hydroxy-4-(3-do-/tri-decyloxy-2-hydroxy-propoxy)-5-α-cumylphenyl]-s-triazine;
(y) reaction product of 2,4,6-tris(2,4-dihydroxyphenyl)-s-triazine with octyl α-haloacetate; or
(z) the mixture of 3,3;3,5;5,5-methylene-bis[2,4-bis(2,4-dimethylphenyl)]-6-[2-hydroxy-4-(3-butyloxy-2-hydroxypropoxyphenyl)]-s-triazine.

15. A composition according to claim 14 wherein the UV absorber is
(a) 4-octyloxy-2-hydroxybenzophenone;
(b) 4-methoxy-2-hydroxybenzophenone;
(d) 2-(2-hydroxy-5-tert-octylphenyl-2H-benzotriazole;
(o) 2-[2-hydroxy-3-(α,α-dimethylbenzyl)-5-tert-octylphenyl]-2H-benzotriazole;
(p) 2-{2-hydroxy-3-tert-butyl-5-[2-(omega-hydroxy-octa(ethyleneoxy)carbonyl)ethyl]-phenyl}-2H-benzotriazole;
(q) 2-{2-hydroxy-3-tert-butyl-5-[2-(octyloxy)carbonyl)ethyl]phenyl}-2H-benzotriazole;
(y) reaction product of 2,4,6-tris(2,4-dihydroxyphenyl)-s-triazine with octyl α-haloacetate; or
(z) the mixture of 3,3;3,5;5,5-methylene-bis[2,4-bis(2,4-dimethylphenyl)]-6-[2-hydroxy-4-(3-butyloxy-2-hydroxypropoxyphenyl)]-s-triazine.

16. A composition according to claim 1 wherein the composition also contains an antioxidant.

17. A composition according to claim 16 wherein the antioxidant is a phenolic antioxidant, phosphite, nitrone, amine oxide or hydroxylamine, or mixture thereof.

18. A composition according to claim 1 wherein the effective amount of UV absorber in combination with the hexahydro-1,4-diazepin-5-one compound and an antioxidant is from about 0.01 to about 10% by weight based on the wax.

19. A composition according to claim 18 wherein the effective amount of UV absorber in combination with the hexahydro-1,4-diazepin-5-one compound and an antioxidant is from about 0.1 to about 2% by weight based on the wax.

20. A composition according to claim 19 wherein the effective amount of UV absorber in combination with the hexahydro-1,4-diazepin-5-one compound and an antioxidant is from about 0.1 to about 0.5% by weight based on the wax.

21. A composition according to claim 16 wherein the antioxidant is
n-octadecyl 3,5-di-tert-butyl-4-hydroxyhydrocinnamate,
neopentanetetrayl tetrakis(3,5-di-tert-butyl-4-hydroxyhydrocinnamate),
di-n-octadecyl 3,5-di-tert-butyl-4-hydroxybenzylphosphonate,
1,3,5-tris(3,5-di-tert-butyl-4-hydroxybenzyl)isocyanurate,
thiodiethylene bis(3,5-di-tert-butyl-4-hydroxyhydrocinnamate),
1,3,5-trimethyl-2,4,6-tris(3,5-di-tert-butyl-4-hydroxybenzyl)benzene,
3,6-dioxaoctamethylene bis(3-methyl-5-tert-butyl-4-hydroxyhydrocinnamate),
2,6-di-tert-butyl-p-cresol,
2,2′-ethylidene-bis(4,6-di-tert-butylphenol),
1,3,5-tris(2,6-dimethyl-4-tert-butyl-3-hydroxybenzyl)isocyanurate,
1,1,3,-tris(2-methyl-4-hydroxy-5-tert-butylphenyl)butane,
1,3,5-tris[2-(3,5-di-tert-butyl-4-hydroxyhydrocinnamoyloxy)ethyl]isocyanurate,
3,5-di-(3,5-di-tert-butyl-4-hydroxybenzyl)mesitol,
hexamethylene bis(3,5-di-tert-butyl-4-hydroxyhydrocinnamate),
1-(3,5-di-tert-butyl-4-hydroxyanilino)-3,5-di(octylthio)-s-triazine,
N,N′-hexamethylene-bis(3,5-di-tert-butyl-4-hydroxyhydrocinnamamide),
calcium bis(ethyl 3,5-di-tert-butyl-4-hydroxybenzylphosphonate),
ethylene bis[3,3-di(3-tert-butyl-4-hydroxyphenyl)butyrate],
octyl 3,5-di-tert-butyl-4-hydroxybenzylmercaptoacetate,
bis(3,5-di-tert-butyl-4-hydroxyhydrocinnamoyl)hydrazide,
N,N-di-(C₁₄-C₂₄alkyl)-N-methylamine oxide, or
N,N-dialkylhydroxylamine prepared from di(hydrogenated tallow)amine by direct oxidation.

22. A composition according to claim 21 wherein the antioxidant is
neopentanetetrayl tetrakis(3,5-di-tert-butyl-4-hydroxyhydrocinnamate),
n-octadecyl 3,5-di-tert-butyl-4-hydroxyhydrocinnamate,
1,3,5-trimethyl-2,4,6-tris(3,5-di-tert-butyl-4-hydroxybenzyl)benzene,
1,3,5-tris(3,5-di-tert-butyl-4-hydroxybenzyl)isocyanurate,
2,6-di-tert-butyl-p-cresol, or
2,2′-ethylidene-bis(4,6-di-tert-butylphenol).

23. A hexahydro-1,4-diazepin-5-one compound of formula (III) or (IV) 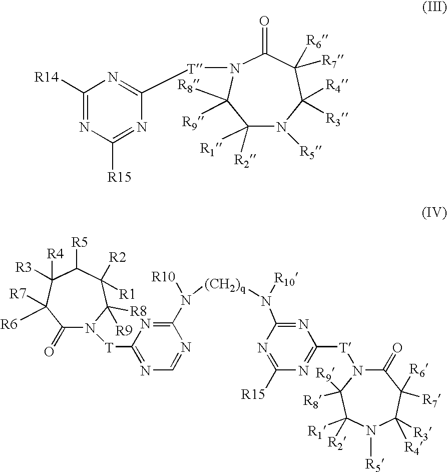
wherein
n is 1 or 2,
p is 1 to 12,
when n is 1,
G is hydrogen, alkyl of 1 to 18 carbon atoms, alkyl of 2 to 18 carbon atoms substituted by NH₂, by OH, by halogen, by —OR₁₀, by 4-hydroxy-3,5-di-tert-butylphenyl or by 4-hydroxy-3,5-dimethyl-phenyl, or by a mixture of these groups; or G is alkyl of 2 to 18 carbon atoms interrupted by —O—, by —CO—, by —NH— or by —NR₁₀— or by a mixture of said groups; or G is alkyl of 1 to 18 carbon atoms substituted by —COOR₁₁ or by phenyl;
when n is 2,
G is alkylene of 2 to 10 carbon atoms, alkenylene of 4 to 12 carbon atoms, alkylene of 3 to 15 carbon atoms substituted by one to four OH or said alkylene interrupted by one to four oxygen atoms; or G is phenylene, C₁-C₄alkylphenylene, —CO—(CH₂)ₚ—CO—, or —COO—;
R₁, R₂, R₃ and R₄ are independently alkyl of 1 to 12 of carbon atoms, said alkyl substituted by one or two hydroxyl groups; or R₁ and R₂ together or R₃ and R₄ together or R₁ and R₂ together and R₃ and R₄ together are tetramethylene, pentamethylene or pentamethylene substituted by methyl;
R₁′, R₂′, R₃′ and R₄′ and R₁″, R₂″, R₃″ and R₄″ are defined independently as for R₁, R₂, R₃ and R₄;
R₆, R₇, R₈ and R₉ are independently hydrogen, alkyl of 1 to 12 of carbon atoms, said alkyl substituted by one or two hydroxyl groups; or R₆ and R₇ together or R₈ and R₉ together or R₆ and R₇ together and R₈ and R₉ together are tetramethylene, pentamethylene or pentamethylene substituted by methyl;
R₆′, R₇′, R₈′ and R₈′ and R₆″, R₇″, R₈″ and R₉″ are defined independently as for R₆, R₇, R₈, and R₉;
R₅is hydrogen, oxyl, hydroxyl, cyanoethyl, phenylalkyl of 7 to 15 carbon atoms, said phenylalkyl substituted on the phenyl ring by one to three radicals selected from alkyl of 1 to 4 carbon atoms and alkoxy of 1 to 4 carbon atoms, phenylalkoxy of 7 to 15 carbon atoms, said phenylalkoxy substituted on the phenyl ring by one to three radicals selected from alkyl of 1 to 4 carbon atoms and alkoxy of 1 to 4 carbon atoms, cycloalkoxy of 5 to 12 carbon atoms, alkenyl of 3 to 8 carbon atoms, alkynyl of 3 to 8 carbon atoms, alkoxy of 1 to 24 carbon atoms, alkyl of 1 to 24 carbon atoms, said alkyl substituted by one or two hydroxyl groups, said alkyl substituted by alkylcarbonyloxy of 2 to 18 carbon atoms, or said alkyl interrupted by one to ten oxygen atoms and substituted by one hydroxyl, by alkylcarbonyloxy of 2 to 18 carbon atoms or by 4-hydroxy-3,5-di-tert-butylbenzoyloxy; or R₅is alkanoyl of 1 to 8 carbon atoms, alkenoyl of 3 to 5 carbon atoms, alkanoyloxy of 1 to 18 carbon atoms, glycidyl, or a group —CH₂ CH(OH)-E where E is hydrogen, methyl or phenyl; T, T′, and T″ are independently defined as alkylene of 1 to 12 carbon atoms; said alkylene interrupted by one to four oxygen atoms; said alkylene interrupted by one to four —NH— groups or —N(R₁₀)— groups; said alkylene interrupted by —OC(O)—, —C(O)—, —C(O)N(H)—, or —C(O)N(R₁₀)—; said alkylene substituted by one to four hydroxyl groups; said alkylene substituted by one to four —NH₂ groups or —N(R₁₀)H groups or —N(R₁₀)(R₁₃) groups; or T, T′, and T″ are independently —CH₂—CH(R₁₂)—O— wherein R₁₂ is hydrogen, methyl or phenyl, or T, T′, and T″ are independently —(CH₂)₃—NH— or T, T′, and T″ are independently a direct bond;
R₅′ and R₅″ are defined as for R₅;
R₁₀ and R₁₀′ are independently alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;
R₁₃ is alkyl of 1 to 8 carbon atoms, alkanoyl of 1 to 8 carbon atoms, cycloalkyl of 5 to 12 carbon atoms, phenylalkyl of 7 to 15 carbon atoms which is unsubstituted or substituted on the phenyl ring by alkyl of 1 to 4 carbon atoms, by alkoxy or 1 to 4 carbon atoms or by a mixture of said groups; or said alkyl of 1 to 8 carbon atoms or said alkanoyl of 1 to 8 carbon atoms substituted by —OH, by alkoxy of 1 to 12 carbon atoms, by benzophenonyl or benzophenonyloxy where one or both phenyl rings of the benzophenone moiety are unsubstituted or substituted by OH, by halogen, by alkyl of 1 to 4 carbon atoms, by alkoxy of 1 to 4 carbon atoms or by a mixture of such groups;
R₆′ and R₆″ are defined as for R₆;
R₁₁ and R₁₁′ are independently hydrogen, alkyl of 1 to 18 carbon atoms, alkyl of 2 to 18 carbon atoms which is substituted by —NH₂, by —NHR₁₀, by —N(R₁₀)₂, by nitro, by hydroxy, by alkoxy of 1 to 18 carbon atoms or by a mixture of said groups; or R₁₁ and R₁₁′ are alkenyl of 3 to 18 carbon atoms, cycloalkyl of 5 to 12 carbon atoms, or said cycloalkyl which is substituted by alkyl of 1 to 4 carbon atoms or is interrupted by —O—, or R₁₁ and R₁₁′ is phenyl or phenylalkyl of 7 to 15 carbon atoms;
R₁₄ is —N(R₁₆)(R₁₆′) or is chlorine, alkoxy of 1 to 12 carbon atoms, thioalkyl of one to eighteen carbon atoms, 2-hydroxyethylamino or —N(R₁₁)(R₁₁′);
or R₁₄ is 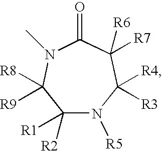
R₁₅ is defined as for R₁₄;
R₁₆ and R₁₆′ are independently glycidyl, C₂-C₁₂alkanoyl substituted by a di(C₁-C₆alkyl) phosphonate, or
R₁₆ and R₁₆′ are independently C₁-C₁₂alkyl, C₂-C₁₂alkanoyl or C₇-C₁₈phenylalkyl, each interrupted by one to six oxygen, sulfur or —N(R₁₀)— groups; C₁-C₁₂alkyl, C₂-C₁₂alkanoyl, phenyl or C₇-C₁₈phenylalkyl, each substituted by one to six hydroxy groups or by one to six —NHR₁₀ groups; C₂-C₁₂alkyl, C₂-C₁₂alkanoyl or C₇-C₁₈phenylalkyl, each interrupted by one to three —NR₁₀C(O)— groups; or R₁₆ and R₁₆′ are independently C₁-C₁₂alkyl, C₂-C₁₂alkanoyl, phenyl or C₇-C₁₈phenylalkyl, each substituted by one to three —SO₃H groups or by one to three —COOR₁₁ groups; and
q is 2to 8.

24. A hexahydro-1,4-diazepin-5-one compound according to claim 23 selected from the group consisting of
(a) 1,3,5-tris(1-cyclohexyl-2,2,7,7-tetramethylhexahydro1,4-diazepin-5-on-4-yl)-s-triazine;
(b) 1,3,5-tris(1,2,2,7,7-pentamethylhexahydro1,4-diazepin-5-on-4-yl)-s-triazine;
(c) 1,3-bis(1-cyclohexyloxy-2,2,7,7-tetramethylhexahydro1,4-diazepin-5-on-4-yl)-5-(2-hydroxylethyl)amino-s-triazine;
(d) 1,3-bis(1-cyclohexyl-2,2,7,7-tetramethylhexahydro1,4-diazepin-5-on-4-yl)-5-(2-hydroxylethyl)amino-s-triazine;
(e) 1-(1-octyloxy-2,2,7,7-tetramethylhexahydro1,4-diazepin-5-on-4-yl)-3,5-bis(2-hydroxylethyl)amino-s-triazine; and
(f) 1-(1-methoxy-2,2,7,7-tetramethylhexahydro1,4-diazepin-5-on-4-yl)-3,5-bis(methylthio)-s-triazine.

25. A composition which comprises
(c) an organic material subject to the deleterious effects of light, heat and oxygen, and
(d) an effective stabilizing amount of a compound of formula (III) and (IV) according to claim 23.
